(19) United States
(12) Patent Application Publication
(10) Pub. No.: US 2004/0266811 A1
(43) Pub. Date: Dec. 30, 2004

(54) Methods for inducing regeneration, remyelination, and hypermyelination of nervous tissue

(76) Inventors: David E. Weinstein, Dobbs Ferry, NY (US); Scott Wadsworth, New Hope, PA (US); John Siekierka, Dobbs Ferry, NY (US); Raymond Birge, New York, NY (US)

Correspondence Address: Leslie Gladstone Restaino, Esq., Brown Raysman Millstein Felder & Steiner LLP, 163 Madison Avenue, P.O. Box 1989, Morristown, NJ, 07962-1989, US

(21) Appl. No.: 10/290,654

(22) Filed: Nov. 8, 2002

(51) Int. Cl. A61K 31/4745

(52) U.S. Cl. 514/291

(57) ABSTRACT

The present invention provides methods for enhancing regeneration or remyelination, and for inducing hypermyelination, of neurites in damaged nervous tissue. Additionally, the present invention provides a method for modulating gene expression in Schwann cells. The present invention is further directed to uses of immunophilin ligands to enhance regeneration or remyelination of neurites in damaged nervous tissue, to induce hypermyelination of neurites in damaged nervous tissue, and to modulate gene expression in Schwann cells. Also provided is a pharmaceutical composition, comprising GM-284 and a pharmaceutically acceptable carrier. The present invention is also directed to methods for treating a peripheral neuropathy in a subject in need of treatment. Finally, the present invention provides a use of GM-284 to treat a peripheral neuropathy in a subject in need of treatment.

### INCORPORATION BY REFERENCE

This application hereby incorporates by reference, in its entirety, U.S. patent application entitled, “METHODS FOR PROMOTING WOUND HEALING AND USES THEREOF”, attorney docket no. 5402/4, application Ser. No. 10/______, concurrently filed on Nov. 8, 2002.

### BACKGROUND OF THE INVENTION

Following mechanical transection of peripheral nerves, axons contained within the transected nerve bundle are capable of regenerating with a high degree of fidelity, such that they find their original target fields and re-establish functional synapses. Nerve injuries in which the nerve sheath remains intact result in near-complete recovery (Dyck et al., “Pathological Alterations of Nerves”, in Peripheral Neuropathy, P. J. Dyck and P. K. Thomas, eds. (Philadelphia, Pa.: W. B. Saunders Company, 1993) pp. 514-95). During the regenerative process, Schwann cells in the distal stump of the transected nerve act to draw axons down the remaining endoneurial tubes, and then remyelinate the appropriately-sized fibers (Weinstein, D. E., The role of Schwann cells in neural regeneration. The Neuroscientist, 5:208-16, 1999). Thus, following a simple nerve injury, regeneration of axons and myelin is both an elegant, and an exceptionally slow, process.

Under optimized conditions, axons regenerate at a rate of 1 nm/day. This rate decreases with increasing age of the individual, and increasing length of the segment requiring regeneration (Griffin and Hoffman, “Degeneration and Regeneration in the Peripheral Nervous System”, in Peripheral Neuropathy, P. J. Dyck and P. K. Thomas, eds. (Philadelphia, Pa.: W. B. Saunders Company, 1993) pp. 361-76). For example, following crushing of the proximal segments of the sciatic nerve in an adult human, it will likely take years for the distal motor and sensory targets in the foot to fully reanimate, if ever. Following other types of nerve injury, in which there is partial or complete disconnection of the proximal and distal stumps of the nerve, regeneration is severely impaired.

Without surgical treatment, peripheral nerve injuries result in either partial regeneration, misdirected axons with the potential of synkinesis, or an absence of regeneration with or without neuroma formation (Yamamoto et al., Occurrence of sequelae in Bell's palsy. Acta. Otolaryngol. Suppl., 446:93-96, 1988; Pavesi et al., Unusual synkinetic movements between facial muscles and respiration in hemifacial spasm. Mov. Disord., 9:451-44, 1994; Strauch et al., The generation of an artificial nerve, and its use as a conduit for regeneration. J. Reconstr. Microsurg., 17:589-98, 2001; Strauch et al., Determining the maximal length of a vein conduit used as an interposition graft for nerve regeneration. J. Reconstr. Microsurg., 12:521-57,1996). Accordingly, in view of the inherent limitations of the regenerating nervous system, as well as the clinical necessities resulting from impaired motor and sensory function in patients with neural injury, there exists a need to investigate both pharmacological and surgical interventions that may augment and/or accelerate peripheral nerve regeneration.

Major advances in pharmacological therapies that induce heightened nerve regeneration have centered on the fortuitous observation that the immunosuppressive drug, FK506 (tacrolimus), promotes nerve regeneration (Gold, B. G., FK506 and the role of immunophilins in nerve regeneration. Mol. Neurobiol., 15:285-306, 1997; Jost et al., Acceleration of peripheral nerve regeneration following FK506 administration. Restor. Neurol. Neurosci., 17:39-44, 2000). However, neither the molecular biological mechanism(s) by which FK506 and related compounds exert their proregenerative activities, nor the cells that respond to the effects of FK506 and related compounds, have been defined.

It is known that the immunomodulatory activities of both FK506 and cyclosporin—the other major immunosuppressive drug used in solid-organ transplantation—are exerted through tight binding of these molecules to the calmodulin-dependent senne/threonine protein phosphatase, calcineurin. Additionally, it is known that, in T cells, the binding of either FK506 or cyclosporin to calcineurin impinges upon signaling pathways to prevent immunoactivation and IL-2 production, thereby blocking the cellular immune cascade early in the process (Harding et al., A receptor for the immunosuppressant FK506 is a cis-trans peptidyl-prolyl isomerase. Nature, 341:758-60, 1989; Liu et al., Calcineurin is a common target of cyclophilin-cyclosporin A and FKBP-FK506 complexes. Cell, 66:807-15, 1991; Schreiber, S. L., Immunophilin-sensitive protein phosphatase action in cell signaling pathways. Cell, 70:365-68, 1992; Siekierka and Sigal, FK-506 and cyclosporin A: immunosuppressive mechanism of action and beyond. Curr. Opin. Immunol., 4:548-52, 1992). Furthermore, it is recognized that FK506 binds with high affinity to endogenous intracellular receptors, called immunophilins (Kay, J. E., Structure-function relationships in the FK506-binding protein (FKBP) family of peptidylprolyl cis-trans isomerases. Biochem. J, 314:361-85, 1996), which can be further segregated into two distinct families, FK506-binding proteins (FKBPs) and cyclophilins.

Immunophilins are ubiquitously-expressed proteins with peptidyl-proline cis/trans isomerase activity (Galat and Metcalfe, Peptidylproline cis/trans isomerases. Prog. Biophys. Mol. Biol., 63:67-118, 1995; Marks, A. R., Cellular functions of immunophilins. Physiol. Rev., 76:631-49, 1996). The calcineurin- and immunophilin-binding capacities of FK506 are separable, and reside in different domains of the molecule. Moreover, it is very likely that the proneuroregenerative activity of FK506 lies outside the calcineurin-binding activity, as cyclosporin has the same activity, but shows little ability in actively promoting neural regeneration (Jost et al., Acceleration of peripheral nerve regeneration following FK506 administration. Restor. Neurol. Neurosci., 17:39-44, 2000).

Analysis of the FK506 molecule and its intracellular binding proteins has yielded considerable insight into the pharmacology of the compound, and has led to suggested models for the generation of mimetic compounds that maintain the neural-promoting activity of FK506 while simultaneously dispensing with the immunosuppressive moieties of the drug. In particular, the foregoing observations have led to the synthesis of a series of compounds known as the nonimmunosuppressive immunophilin ligands. Among these compounds are the Vertex drug, V10, 367, and the Guilford compound, GPI-1046. These FK506 mimetics neither bind to, nor inhibit, calcineurin; therefore, they lack immunosuppressive activity, but retain the proneuroregenerative activities of the parent compound (Steiner et al., Neurotrophic immunophilin ligands stimulate structural and functional recovery in neurodegenerative animal models. Proc. Natl. Acad. Sci. USA, 94:2019-24, 1997; Hamilton and Steiner, Immunophilins: beyond immunosuppression. J. Med. Chem., 41:5119-43, 1998).

Studies intended to elucidate the proregenerative mechanism(s) of action of the immunophilin ligands have suggested that these compounds act indirectly, or in combination with one or more endogenous activities, to promote neurite outgrowth. Specifically, studies using purified chick sensory neurons (PC12 or SH-SY5Y cells) have all shown that FK506, GPI-1046, and V10,367 potentiate neurite outgrowth only when co-administered with sub-threshold concentrations of NGF (Gold, B. G., FK506 and the role of immunophilins in nerve regeneration. Mol Neurobiol., 15:285-306, 1997). Thus, these observations establish that FK506 and its analogues are, by definition, not neurotrophic molecules on their own. Furthermore, these observations suggest that immunophilins are likely to act in the potentiation or induction of endogenous activities that regulate neurite outgrowth and regeneration.

It has previously been shown that FK506 can stimulate growth of damaged peripheral nerves or neurons in certain patients, by administering the FK506 directly to the damaged nerves or neurons (see, e.g., U.S. Pat. No. 6,080,753). However, FK506's proregenerative mechanism of action has not previously been known. In particular, prior to the present invention, it was not previously known that FK506 and FK506-related compounds and derivatives mediate neuronal growth indirectly. Furthermore, FK506 is an immunosuppressive; therefore, a compound that can enhance neuron regeneration, while avoiding the immunosuppressive effects of FK506, would be desirable in the treatment of peripheral nerve disease.

### SUMMARY OF THE INVENTION

The present invention is based upon the discovery, disclosed herein, that the neuritogenic actions of FK506-related compounds, particularly nonimmunosuppressive derivatives of FK506, are indirect, as they are mediated through Schwann cells. Using cDNA-array analysis of RNAs from drug-treated Schwann cells, the inventors have identified a series of transcription factors that are upregulated in a temporal cascade. The upregulated genes, SCIP and Bm-5, are members of the POU family of transcription factors. The inventors recently have demonstrated that these gene products regulate the timing and extent of in vivo myelination, and are associated with maintenance of the myelinating state. In addition, the inventors have shown that one of these genes, SCIP, regulates both the rate and extent of axonal regeneration, and the myelin:axon ratio following nerve injury.

In view of the foregoing, the present invention provides a method for enhancing regeneration of a neurite in damaged nervous tissue, by contacting at least one Schwann cell adjacent to the neurite in the damaged nervous tissue with an amount of an immunophilin ligand effective to enhance regeneration of the neurite.

The present invention further provides a use of an immunophilin ligand to regenerate a neurite in damaged nervous tissue, wherein at least one Schwann cell adjacent to the neurite in the damaged nervous tissue is contacted with an amount of the immunophilin ligand effective to enhance regeneration of the neurite.

Additionally, the present invention provides a method for enhancing remyelination of a neurite in damaged nervous tissue, by contacting at least one Schwann cell adjacent to the neurite in the damaged nervous tissue with an amount of an immunophilin ligand effective to enhance remyelination of the neurite.

Also provided is a use of an immunophilin ligand to enhance remyelination of a neurite in damaged nervous tissue, wherein at least one Schwann cell adjacent to the neurite in the damaged nervous tissue is contacted with an amount of the immunophilin ligand effective to enhance remyelination of the neurite.

The present invention is further directed to a method for inducing hypermyelination of a neurite in nervous tissue, by contacting at least one Schwann cell adjacent to the neurite in the nervous tissue with an amount of an immunophilin ligand effective to induce hypermyelination of the neurite.

The present invention also provides a use of an immunophilin ligand to induce hypermyelination of a neurite in nervous tissue, wherein at least one Schwann cell adjacent to the neurite in the nervous tissue is contacted with an amount of the immunophilin ligand effective to induce hypermyelination of the neurite.

The present invention further provides a pharmaceutical composition, comprising GM-284 and a pharmaceutically-acceptable carrier.

The present invention is also directed to a method for modulating gene expression in a Schwann cell, by contacting the Schwann cell with an amount of an immunophilin ligand effective to modulate gene expression in the Schwann cell.

Additionally, the present invention provides a use of an immunophilin ligand to modulate gene expression in a Schwann cell, wherein the Schwann cell is contacted with an amount of the immunophilin ligand effective to modulate gene expression in the Schwann cell.

Also provided is a method for treating a peripheral neuropathy in a subject in need of treatment, comprising modulating expression of a Schwann cell transcription factor in the subject, wherein the Schwann cell transcription factor is selected from the group consisting of SCIP and Bm-5.

The present invention further provides a method for treating a peripheral neuropathy in a subject in need of treatment, by administering to the subject an amount of GM-284 effective to treat the peripheral neuropathy in the subject.

Finally, the present invention provides a use of GM-284 to treat a peripheral neuropathy in a subject in need of treatment, wherein GM-284 is administered to the subject in an amount of GM-284 effective to treat the peripheral neuropathy in the subject.

Additional aspects of the present invention will be apparent in view of the description which follows.

### BRIEF DESCRIPTION OF THE FIGURES

FIG. 1 demonstrates that GM-284 induces neurite outgrowth from DRG explants in a Schwann-cell-dependent manner. (A) DRGs were explanted and cultured alone, (panel i), in the presence of 100 ng/ml NGF (panel ii) or in the presence of 1 μM GM-284 (panel iii), for 96 h. At the end of the culture period, the tissue was fixed and stained for expression of neuron-specific β-III tubulin. There was comparable outgrowth in the NGF and GM-284 treated cultures. (B) Both Schwann cells and neurons are present in DRG explants. To determine if the neuritogenic effects of GM-284 are direct, acting on neurons, or indirect, acting through Schwann cells, sensory neurons from E18 mice were isolated and treated with either NGF (left panel) or GM-284 (right panel) for 6 days. There was no neurite outgrowth in purified neuronal cultures treated with GM-284; however, sister cultures responded to NGF.

FIG. 2 illustrates that GM-284-induced neurite outgrowth is NGF- and MEK1-independent. (A) DRGs were prepared and cultured as above, except that either the MEK1 inhibitor, PD 098059, or NGF-neutralizing antibodies were included in cultures treated with either NGF or GM-284. Maximal response was determined to be the extent of neurite outgrowth when DRGs were cultured in NGF alone (spotted bars) or in 1 μM GM-284 (striped bars). As indicated, DRGs were treated with either NGF or GM-284, in the presence of either 0.25 mg/ml anti-NGF neutralizing antibodies or PD 098059. (B) GM-284 fails to activate the ERK pathway in DRGs. DRGs were treated with 100 ng/ml NGF or 1 μM GM-284, for 30 min or 4 h, as indicated, and the detergent lysates were prepared and immunoblotted with anti-phospho-Erk monoclonal antibody. The p42 and p44 bands are indicated by arrowheads.

FIG. 3 shows that GM-284 is an immunophilin ligand. (A) The disassociation constant (kd) of FK506 and of GM-284, as a measure of binding affinity for recombinant FKBP52, was determined by solution-phase tryptophan fluorescence (QTFS), as described below. The panel on the left shows the percent of fluorescence intensity when FK506 is bound to immobilized FKBP52, yielding a kd of 269+50.8. The right panel shows the percent fluorescence after GM-284 binding to FKBP52, demonstrating a kd of 139±16.2. There was virtually no binding of GST alone to FKBP52 (not shown). (B) GM-284 competes with FK506 for binding to full-length FKBP52. Full-length FKBP52 was expressed as a GST fusion protein, purified over GSH-Sepharose resin. The GST-FKBP52 (1 μg) was immobilized on GSH-Sepharose beads, and incubated with 10 nM FK506 containing 1.5 μCi/ml ³H-dihydro-FK506 (black bars) alone, or in the presence of 200-fold molar excess of cold FK506 (upper) or GM-284 (lower). After extensive washing, the bound fraction (gray bars) was determined by liquid scintillation counting. The immunoblots of FKBP52 demonstrate that equal concentrations of receptor were bound for each experimental point, and that the receptor was not lost with extensive washing. Values are averages (with standard deviations) of three independent experiments.

FIG. 4 illustrates that Schwann cells are critical for the neuritogenic effects of GM-284. (A) Isolation of primary Schwann cells from sciatic nerve. Primary Schwann cells, cultured in DMEM containing serum, forskolin, and GGF (panel i), were seeded onto chamber slides and stained with anti-S-100β, followed by a secondary fluorescein-conjugated IgG (panel ii). Cell nuclei were counter-stained with Hoechst stain (panel iii). Intense S-100β fluorescence staining was observed in a majority of the cells, indicating an almost pure population of Schwann cells in the cultures. Staining of the Schwann cells with an anti-FKBP52 mAb showed uniform cytoplasmic staining (panel iv). In contrast, there was little or no background in the absence of the primary antibody (panel v). The inset in panel (iv) shows the results of Western blotting of Schwann cells with the same anti-FKBP52 mAb (lane 2) or no primary antibody (lane 1). (B) GFP-expressing PC12 cells were co-cultured on monolayers of Schwann cells cultured with either DMSO vehicle (panel a), 1 μM GM-284 (panel b), or 100 ng/ml NGF (panel c) for 72 h. Thereafter, the extent of neurite outgrowth was determined by observing GFP fluorescence (panels d-f). To determine if the Schwann-cell-dependent outgrowth activity observed was contact-dependent, or recoverable in the media, conditioned media from purified Schwann cells grown in the absence (panel e) or presence (panel f) of 1 μM GM-284 were collected and added to TrkA-overexpressing PC12 cells, as described below. Neurite promotion was observed only in the GM-284-treated Schwann cells' conditioned media (CM).

FIG. 5 demonstrates that GM-284 induces Schwann cell expression of SCIP (Oct-6) and Brn-5. Two members of the POU family of transcription factors are among the known genes upregulated at 48 h after Schwann cells are treated with GM-284. To further assess the ability of GM-284 to regulate these genes, Schwann cells were maintained in DMEM containing FCS, and without forskolin or GGF, for 48 h, to rest the cells. Thereafter, 1 μM GM-284 was added for an additional 48 h. Total RNA was isolated, run on a denaturing agarose gel, transferred to a nylon filter, and hybridized with probes complimentary to either SCIP (panel a) or Brn-5 (panel b). Cyclophilin RNA was probed as a loading control.

FIG. 6 shows that GM-284 augments peripheral nerve regeneration after mechanical transection. (A) The sciatic nerves of adult, outbred ICR mice were crushed, as previously described (Gondre et al., Accelerated nerve regeneration mediated by Schwann cells expressing a mutant form of the POU protein SCIP. J. Cell Biol., 141:493-501, 1998). Following the surgery, the animals were randomized into treatment groups of either GM-284 (10 mg/kg) or vehicle. At the end of 30 days, the animals were sacrificed, their sciatic nerves were prepared for electron microscopy, and subsequently evaluated for the extent of both axonal and myelin regeneration. All sampling was done at the same proximal-distal levels, and approximately 5 mm below the crush site. In comparison with the vehicle-treated sciatic nerves (panel a), the GM-284-treated tissue (panel b) showed clear axonal hypertrophy, as well as hypermyelination. (B) Quantitative analysis of both axonal and myelin growth in response to GM-284 treatment was carried out by digitizing the images in (A), and determining the axonal and myelin volumes, calculated in voxels. GM-284 treatment resulted in a ˜3-fold increase in myelin (left panel), and a 4-fold increase in the size of the associated axons (right panel), following one month of treatment.

FIG. 7 illustrates that GM-284 treatment of nerve injury phenocopies overexpression of the POU protein SCIP. As described above, GM-284 induces expression of the POU proteins, SCIP and Bm-5, in Schwann cells. Comparison of untreated sciatic nerve (panel a), ASCIP-treated sciatic nerve (panel b), and GM-284-treated nerve (panel c) demonstrates that GM-284 treatment results in hypermyelination and axonal hypertrophy that are indistinguishable from regenerated nerve in animals expressing a dominant-active form of SCIP.

FIG. 8 depicts the structure of GM-284.

### DETAILED DESCRIPTION OF THE INVENTION

Nerve regeneration is a process involving an extremely complex series of interactions that depend upon the cellular interactions of neurons and Schwann cells, and the contribution of blood-borne and basal-lamina-contributed molecules (Weinstein, D. E., The role of Schwann cells in neural regeneration. The Neuroscientist, 5:208-216, 1999). The inventors have recently reported on the absolute requirement for Schwann cells to be present in the nerve to achieve all but minimal axonal regeneration across gaps in the shaft of peripheral nerves (Strauch et al., The generation of an artificial nerve, and its use as a conduit for regeneration. J. Reconstr. Microsurg., 17:589-98, 2001). Moreover, the inventors have shown that Schwann cells can augment both the rate and extent of axonal recovery, as well as myelin recovery, following crushing nerve injury (Gondre et al., Accelerated nerve regeneration mediated by Schwann cells expressing a mutant form of the POU protein SCIP. J. Cell Biol., 141:493-501, 1998). Therefore, given the indirect actions of the immunophilin ligands on neurite outgrowth, and the requirement for Schwann cells to be present in regenerating nerves, the inventors hypothesized that the immunophilin ligand compounds were acting through Schwann cells in promoting regeneration.

As reported herein, the inventors tested their hypothesis by investigating neuritogenic and neural regenerative properties of a novel immunophilin ligand, GM-284. GM-284 is a nonimmunosuppressive immunophilin ligand and an FK506 mimetic. It neither binds to, nor inhibits, calcineurin; therefore, it lacks immunosuppressive activity, but retains the proneuroregenerative activities of the parent compound. As demonstrated herein, GM-284 binds to the immunophilin, FKBP52, which is present in Schwann cells, and potently promotes neurite outgrowth, both in vivo and in vitro. Furthermore, the inventors have shown that the proregenerative activities of this compound, as well as FK506 and its derivatives, are indirect, acting through the Schwann cell. While it was previously known that FK506 can stimulate growth of damaged peripheral nerves or neurons in certain patients, FK506's proregenerative mechanism of action had not been previously established. Finally, the inventors have demonstrated that GM-284's effects on the Schwann cell are mediated at the transcriptional level, upregulating a series of transcription factors that mediate the ability of the Schwann cell to drive axonal regeneration following injury, and that are part of the myelination cascade, both before and after injury.

In view of the foregoing, the present invention provides a method for enhancing regeneration of a neurite in damaged nervous tissue. As used herein, the term “enhancing regeneration of a neurite” means augmenting, improving, or increasing partial or full growth or regrowth of a neurite that has degenerated. As further used herein, the term “growth” refers to an increase in diameter, length, mass, and/or thickness of a neurite, a neuron, or myelin, as the case may be. Causes of neurite degeneration include damage to nervous tissue, death of neurons, demyelination, injury, and various pathologies. Regeneration of the neurite may take place in neurites of both the central nervous system and the peripheral nervous system. Regeneration, and enhanced regeneration, of neurites may be measured or detected by known procedures, including Western blotting for myelin-specific and axon-specific proteins, electron microscopy in conjunction with morphometry, and any of the methods, molecular procedures, and assays disclosed herein.

As used herein, the term “nervous tissue” includes the nervous tissue present in both the central nervous system and the peripheral nervous system, and comprises any or all of the following: axons, dendrites, fibrils, fibular processes, ganglion cells, granule cells, grey matter, myelin, neuroglial cells, neurolimma, neuronal cells or neurons, Schwann cells, stellate cells, and white matter. As further used herein, a “neuron” is a conducting or nerve cell of the nervous system that typically consists of a cell body (perikaryon) that contains the nucleus and surrounding cytoplasm; several short, radiating processes (dendrites); and one long process (the axon), which terminates in twig-like branches (telodendrons), and which may have branches (collaterals) projecting along its course. Examples of neurons include, without limitation, autonomic neurons, neurons of the dorsal root ganglia (DRG), enteric neurons, interneurons, motor neurons, peripheral neurons, sensory neurons, and neurons of the spinal cord. In one embodiment of the present invention, the damaged nervous tissue comprises damaged peripheral neurons.

Additionally, as used herein, the term “neurite” refers to processes of neuronal cells, and includes axons and dendrites. For example, the neurite of the present invention may be a process extending from a neuron, such as an autonomic neuron, a neuron of the dorsal root ganglia (DRG), an enteric neuron, an interneuron, a motor neuron, a peripheral neuron, a sensory neuron, or a neuron of the spinal cord. Thus, the neurite may be, for example, an autonomic neuron neurite, a DRG neurite, an enteric neuron neurite, an interneuron neurite, a motor neuron neurite, a peripheral neuron neurite, a sensory neuron neurite, or a neurite of the spinal cord. In one embodiment of the present invention, the neurite is a peripheral neuron neurite.

As disclosed herein, the method of the present invention comprises contacting at least one Schwann cell adjacent to (e.g., near to and/or in contact with) a neurite in damaged nervous tissue with an immunophilin ligand, in an amount effective to enhance regeneration of the neurite. Immunophilins are ubiquitously-expressed proteins with peptidyl-proline cisltrans isomerase activity (Galat and Metcalfe, Peptidylproline cis/trans isomerases. Prog. Biophys. Mol. Biol., 63:67-118, 1995; Marks, A. R., Cellular functions of immunophilins. Physiol. Rev., 76:631-49, 1996). As endogenous intracellular receptors (Kay, J. E., Structure-function relationships in the FK506-binding protein (FKBP) family of peptidylprolyl cis-trans isomerases. Biochem. J, 314:361-85, 1996), immunophilins can be further segregated into two distinct families: FK506-binding proteins (FKBPs) and cyclophilins.

Unless otherwise indicated, an “immunophilin ligand” is an agent that is reactive with an immunophilin. As used herein, “reactive” means the agent has affinity for, binds to, or is directed against an immunophilin. As further used herein, an “agent” shall include a protein, polypeptide, peptide, nucleic acid (including DNA or RNA), antibody, Fab fragment, F(ab′)₂ fragment, molecule, antibiotic, drug, compound, and any combination thereof. A Fab fragment is a univalent antigen-binding fragment of an antibody, which is produced by papain digestion. An F(ab′)₂ fragment is a divalent antigen-binding fragment of an antibody, which is produced by pepsin digestion. Additionally, as used herein, the term “immunophilin ligand” refers to immunophilin ligands and any analogues and derivatives thereof, including, for example, a natural or synthetic functional variant of an immunophilin ligand. Preferably, the immunophilin ligand of the present invention is a small molecule that binds an immunophilin receptor.

It is recognized that FK506 binds with high affinity to immunophilins (Kay, J. E., Structure-function relationships in the FK506-binding protein (FKBP) family of peptidylprolyl cis-trans isomerases. Biochem. J, 314:361-85, 1996). FK506 (tacrolimus) (Fujisawa Pharmaceutical Co., Ltd, Osaka, Japan) is an immunosuppressive drug that promotes nerve regeneration (Gold, B. G., FK506 and the role of immunophilins in nerve regeneration. Mol. Neurobiol., 15:285-306, 1997; Jost et al., Acceleration of peripheral nerve regeneration following FK506 administration. Restor. Neurol. Neurosci., 17:39-44, 2000). A series of compounds, known as the nonimmunosuppressive immunophilin ligands, have been synthesized on the basis of FK506. Among these compounds are the Vertex drug, V10,367 (Vertex Pharmaceuticals, Cambridge Mass.), the Guilford compound, GPI-1046 (Guilford Pharmaceuticals, Baltimore, Md.), and a novel nonimmunosuppressive ligand disclosed herein, termed GM-284. These FK506 mimetics neither bind to, nor inhibit, calcineurin; therefore, they lack immunosuppressive activity, but retain the proneuroregenerative activities of the parent compound (Steiner et al., Neurotrophic immunophilin ligands stimulate structural and functional recovery in neurodegenerative animal models. Proc. Natl. Acad. Sci. USA, 94:2019-24, 1997; Hamilton and Steiner, Immunophilins: beyond immunosuppression. J. Med. Chem., 41:5119-43, 1998).

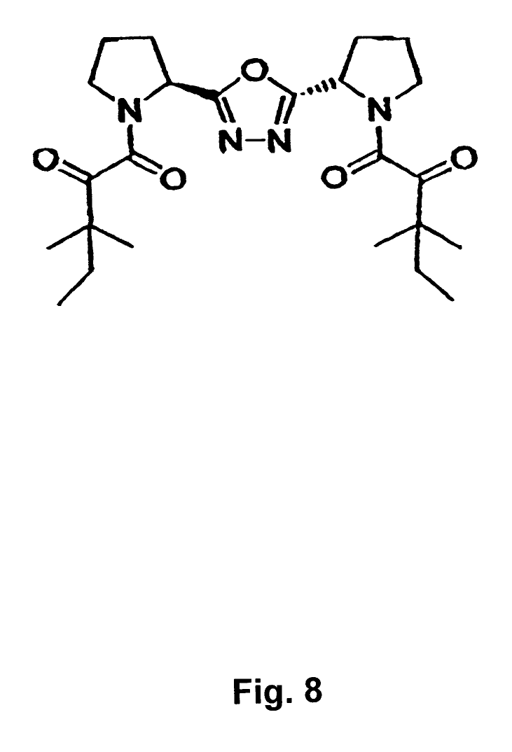

Accordingly, in one embodiment of the present invention, the immunophilin ligand is FK506 or an FK506 analogue or derivative. As used herein, an “FK506 derivative” is a chemical substance derived from FK506, either directly or by modification, truncation, or partial substitution. FK506 and its analogues and derivatives may be produced synthetically. The FK506 derivative for use in the present invention may be nonimmunosuppressive. In a preferred embodiment of the present invention, the nonimmunosuppressive FK506 derivative is GM-284. This novel compound is a small molecule that effects transcriptional change in Schwann cells, as described below. GM-284 is an immunophilin ligand; its disassociation constant (k_{d}), as a measure of binding affinity for recombinant FKBP52, and as determined by solution-phase tryptophan fluorescence (QTFS), is 139±16.2. The structure of GM-284 is depicted in FIG. 8. Additionally, it can be found as compound 30 in international application no. PCT/US00/16221 (publication no. WO 01/04116), which is herein incorporated by reference, and prepared in accordance with methods described in that application. It is believed that GM-284 will be effective as a drug to treat many types of disorders associated with nervous tissue degeneration.

In the method of the present invention, at least one Schwann cell adjacent to a neurite in damaged nervous tissue is contacted with an amount of an immunophilin ligand effective to enhance regeneration of the neurite. This amount may be readily determined by the skilled artisan, based upon known procedures, including analysis of titration curves established in vivo, and methods disclosed herein.

The method of the present invention may be used to enhance regeneration of a neurite in vitro, or in vivo in a subject. For example, an immunophilin ligand may be contacted in vitro with at least one Schwann cell adjacent to a neurite in damaged nervous tissue by introducing the immunophilin ligand to the tissue containing the Schwann cell using conventional procedures. Alternatively, an immunophilin ligand may be contacted in vivo with at least one Schwann cell adjacent to a neurite in damaged nervous tissue in a subject by administering the immunophilin ligand to the subject. The subject may be any animal, but is preferably a mammal (e.g., humans, domestic animals, and commercial animals). More preferably, the subject is a human.

It is also within the confines of the present invention that an immunophilin ligand may be introduced to tissue containing Schwann cells adjacent to neurites in vitro, using conventional procedures, to achieve enhanced regeneration of neurites in vitro. Thereafter, tissue containing regenerated neurites may be introduced into a subject to provide regenerated neurites in vivo. In such an ex vivo approach, the nervous tissue is preferably removed from the subject, subjected to introduction of the immunophilin ligand, and then reintroduced into the subject.

In the method of the present invention, the neurites and the Schwann cell(s) adjacent to the neurites may be contained in damaged neural tissue and other damaged tissue of the nervous system, in vitro or in vivo in a subject, either alone or with other types of neural cells, including, for example, astrocytes, ganglion cells, granule cells (both cerebellar and hippocampal), neuroglial cells, neurons, oligodendroglia, Schwann cells, and stellate cells. Neurons and Schwann cells may be detected in damaged tissue by standard detection methods readily determined from the known art, examples of which include, without limitation, immunological techniques (e.g., immunohistochemical staining), fluorescence-imaging techniques, and microscopic techniques.

The ability of immunophilin ligands, particularly FK506 derivatives such as GM-284, to modulate gene expression in Schwann cells, and thereby enhance regeneration of neurites, renders immunophilin ligands particularly useful for treating conditions associated with nervous tissue degeneration. As used herein, “nervous tissue degeneration” means a condition of deterioration of nervous tissue, wherein the nervous tissue changes to a lower or less functionally-active form. It is believed that, by enhancing neurite regeneration, immunophilin ligands will be useful for the treatment of conditions associated with nervous tissue degeneration. It is further believed that immunophilin ligands, including GM-284, would be effective either alone or in combination with other therapeutic agents that are typically used in the treatment of these conditions.

Accordingly, the present invention provides a method for treating nervous tissue degeneration in a subject in need of treatment, comprising contacting at least one Schwann cell adjacent to a neurite in damaged nervous tissue in the subject with an amount of an immunophilin ligand effective to enhance regeneration of the neurite, thereby treating the nervous tissue degeneration. Nervous tissue degeneration may be caused by, or associated with, a variety of factors, including, without limitation, primary neurologic conditions (e.g., neurodegenerative diseases), central nervous system (CNS) and peripheral nervous system (PNS) traumas, and acquired secondary effects of non-neural dysfunction (e.g., neural loss secondary to degenerative, pathologic, or traumatic events).

Examples of neurodegenerative diseases include, without limitation, Alzheimer's disease, amyotrophic lateral sclerosis (Lou Gehrig's Disease), Binswanger's disease, Huntington's chorea, multiple sclerosis, myasthenia gravis, Parkinson's disease, and Pick's disease. Examples of CNS traumas include, without limitation, blunt trauma, hypoxia, and invasive trauma. Examples of acquired secondary effects of non-neural dysfunction include, without limitation, cerebral palsy, congenital hydrocephalus, muscular dystrophy, stroke, and vascular dementia, as well as neural degeneration resulting from any of the following: an injury associated with cerebral hemorrhage, developmental disorders (e.g., a defect of the brain, such as congenital hydrocephalus, or a defect of the spinal cord, such as spina bifida), diabetic encephalopathy, hypertensive encephalopathy, intracranial aneurysms, ischemia, kidney dysfunction, subarachnoid hemorrhage, trauma to the brain and spinal cord, the treatment of therapeutic agents such as chemotherapy agents and antiviral agents, vascular lesions of the brain and spinal cord, and other diseases or conditions prone to result in nervous tissue degeneration.

Nervous tissue degeneration may arise in the CNS or the PNS. In one embodiment of the present invention, the nervous tissue degeneration of the PNS is a peripheral neuropathy. As defined herein, the term “peripheral neuropathy” refers to a syndrome of sensory loss, muscle weakness, muscle atrophy, decreased deep tendon reflexes, and/or vasomotor symptoms. In a subject who has a peripheral neuropathy, the myelin sheath or Schwann cell may be primarily affected, or the axon may be primarily affected. The peripheral neuropathy may affect a single nerve (mononeuropathy), two or more nerves in separate areas (multiple mononeuropathy), or many nerves simultaneously (polyneuropathy).

Examples of peripheral neuropathies that may be treated by the methods disclosed herein include, without limitation, peripheral neuropathies associated with such conditions as acute or chronic inflammatory polyneuropathy, amyotrophic lateral sclerosis (ALS), collagen vascular disorder (e.g., polyarteritis nodosa, rheumatoid arthritis, Sjögren's syndrome, or systemic lupus erythematosus), diphtheria, Guillain-Barre syndrome, hereditary peripheral neuropathy (e.g., Charcot-Marie-Tooth disease (including type I, type II, and all subtypes), hereditary motor and sensory neuropathy (types I, II, and III, and peroneal muscular atrophy), hereditary neuropathy with liability to pressure palsy (HNPP), infectious disease (e.g., acquired immune deficiency syndrome (AIDS)), Lyme disease (e.g., infection with Borrelia burgdorferi), invasion of a microorganism (e.g., leprosy—the leading cause of peripheral neuropathy worldwide, after neural trauma), leukodystrophy, metabolic disease or disorder (e.g., amyloidosis, diabetes mellitus, hypothyroidism, porphyria, sarcoidosis, or uremia), neurofibromatosis, nutritional deficiencies, peroneal nerve palsy, polio, porphyria, postpolio syndrome, Proteus syndrome, pressure paralysis (e.g., carpal tunnel syndrome), progressive bulbar palsy, radial nerve palsy, spinal muscular atrophy, a toxic agent (e.g., barbital, carbon monoxide, chlorobutanol, dapsone, emetine, heavy metals, hexobarbital, lead, nitrofurantoin, orthodinitrophenal, phenyloin, pyridoxine, sulfonamides, triorthocresyl phosphate, the vinca alkaloids, many solvents, other industrial poisons, and certain AIDS drugs (including didanosine and zalcitabine), trauma (including neural trauma—the leading cause of peripheral neuropathy, worldwide), and ulnar nerve palsy (Beers and Berkow, eds., The Merck Manual of Diagnosis and Therapy, 17^{th} ed. (Whitehouse Station, N.J.: Merck Research Laboratories, 1999) chap. 183). In a preferred embodiment of the present invention, the peripheral neuropathy is ALS or a hereditary peripheral neuropathy.

According to the method of the present invention, an immunophilin ligand may be administered to a human or animal subject by known procedures, including, without limitation, oral administration, parenteral administration (e.g., epifascial, intracapsular, intracutaneous, intradermal, intramuscular, intraorbital, intraperitoneal, intraspinal, intrasternal, intrathecal, intravascular, intravenous, parenchymatous, or subcutaneous administration), sublingual administration, transdermal administration, and administration through an osmotic mini-pump. Preferably, the immunophilin ligand is administered parenterally, by intravenous or subcutaneous injection. The immunophilin ligand of the present invention also may be administered to a subject in accordance with any of the above-described methods for effecting in vivo contact between a target Schwann cell and the immunophilin ligand.

For oral administration, the formulation of the immunophilin ligand may be presented as capsules, tablets, powders, granules, or as a suspension. The formulation may have conventional additives, such as lactose, mannitol, cornstarch, or potato starch. The formulation also may be presented with binders, such as crystalline cellulose, cellulose derivatives, acacia, cornstarch, or gelatins. Additionally, the formulation may be presented with disintegrators, such as cornstarch, potato starch, or sodium carboxymethylcellulose. The formulation also may be presented with dibasic calcium phosphate anhydrous or sodium starch glycolate. Finally, the formulation may be presented with lubricants, such as talc or magnesium stearate.

For parenteral administration (i.e., administration by injection through a route other than the alimentary canal), the immunophilin ligand may be combined with a sterile aqueous solution that is preferably isotonic with the blood of the subject. Such a formulation may be prepared by dissolving a solid active ingredient in water containing physiologically-compatible substances, such as sodium chloride, glycine, and the like, and having a buffered pH compatible with physiological conditions, so as to produce an aqueous solution, then rendering said solution sterile. The formulations may be presented in unit or multi-dose containers, such as sealed ampoules or vials. The formulation may be delivered by any mode of injection, including, without limitation, epifascial, intracapsular, intracranial, intracutaneous, intramuscular, intraorbital, intraperitoneal, intraspinal, intrasternal, intrathecal, intravascular, intravenous, parenchymatous, or subcutaneous.

For transdermal administration, the immunophilin ligand may be combined with skin penetration enhancers, such as propylene glycol, polyethylene glycol, isopropanol, ethanol, oleic acid, N-methylpyrrolidone, and the like, which increase the permeability of the skin to the immunophilin ligand, and permit the immunophilin ligand to penetrate through the skin and into the bloodstream. The ligand/enhancer compositions also may be further combined with a polymeric substance, such as ethylcellulose, hydroxypropyl cellulose, ethylene/vinylacetate, polyvinyl pyrrolidone, and the like, to provide the composition in gel form, which may be dissolved in solvent, such as methylene chloride, evaporated to the desired viscosity, and then applied to backing material to provide a patch. The immunophilin ligand may be administered transdermally at the site in the subject where neural trauma has occurred, or where the nervous tissue degeneration is localized. Alternatively, the immunophilin ligand may be administered transdermally at a site other than the affected area, in order to achieve systemic administration.

The immunophilin ligand of the present invention also may be released or delivered from an osmotic mini-pump or other time-release device. The release rate from an elementary osmotic mini-pump may be modulated with a microporous, fast-response gel disposed in the release orifice. An osmotic mini-pump would be useful for controlling release, or targeting delivery, of the immunophilin ligand.

It is within the confines of the present invention that a formulation containing GM-284 may be further associated with a pharmaceutically-acceptable carrier, thereby comprising a pharmaceutical composition. Accordingly, the present invention further provides a pharmaceutical composition, comprising GM-284 and a pharmaceutically-acceptable carrier. The pharmaceutically-acceptable carrier must be “acceptable” in the sense of being compatible with the other ingredients of the composition, and not deleterious to the recipient thereof. Examples of acceptable pharmaceutical carriers include carboxymethylcellulose, crystalline cellulose, glycerin, gum arabic, lactose, magnesium stearate, methyl cellulose, powders, saline, sodium alginate, sucrose, starch, talc, and water, among others. Formulations of the pharmaceutical composition may be conveniently presented in unit dosage.

The formulations of the present invention may be prepared by methods well known in the pharmaceutical arts. For example, GM-284 may be brought into association with a carrier or diluent, as a suspension or solution. Optionally, one or more accessory ingredients (e.g., buffers, flavoring agents, surface active agents, and the like) also may be added. The choice of carrier will depend upon the route of administration. The pharmaceutical composition would be useful for administering the GM-284 of the present invention to a subject to treat a neurodegenerative disease. The GM-284 is provided in an amount that is effective to treat a neurodegenerative disease, including a peripheral neuropathy, in a subject to whom the pharmaceutical composition is administered. That amount may be readily determined by the skilled artisan, as described above.

The present invention also provides a method for enhancing remyelination of a neurite in damaged nervous tissue. As used herein, the term “enhancing remyelination of a neurite” means augmenting, improving, or increasing partial or full growth or regrowth of the myelin of a neurite that has degenerated. The remyelination of the neurite may take place in the nerves of both the CNS and the PNS. Remyelination, and enhanced remyelination, of neurites may be measured or detected by known procedures, including Western blotting for myelin-specific and axon-specific proteins, electron microscopy in conjunction with morphometry, and any of the methods, molecular procedures, and assays disclosed herein. The method of the present invention comprises contacting at least one Schwann cell adjacent to a neurite in damaged nervous tissue with an immunophilin ligand. The amount of immunophilin ligand that is used is an amount effective to enhance remyelination of a neurite. This amount may be readily determined by the skilled artisan, based upon known procedures, including analysis of in vivo dose curves and measurement of quantities of myelin-specific and axon-specific proteins per unit length of nerve, and methods disclosed herein.

The method of the present invention may be used to enhance remyelination of a neurite in vitro, ex vivo, or in vivo in a subject, in accordance with methods described above. In one embodiment of the present invention, the immunophilin ligand is FK506 or an FK506 derivative. The FK506 derivative for use in the present invention may be nonimmunosuppressive. In a preferred embodiment of the present invention, the nonimmunosuppressive FK506 derivative is GM-284.

It is believed that, by enhancing neurite remyclination, immunophilin ligands will be useful for the treatment of conditions associated with nervous tissue degeneration. It is further believed that immunophilin ligands, including GM-284, would be effective either alone or in combination with other therapeutic agents that are typically used in the treatment of these conditions. Accordingly, the present invention provides a method for treating nervous tissue degeneration in a subject in need of treatment, comprising contacting at least one Schwann cell adjacent to a neurite in damaged nervous tissue in the subject with an amount of an immunophilin ligand effective to enhance remyelination of the neurite, thereby treating the nervous tissue degeneration. Examples of nervous tissue degeneration, including peripheral neuropathies, which may be treated by the method of the present invention are discussed above. In a preferred embodiment of the present invention, the peripheral neuropathy is ALS or a hereditary peripheral neuropathy.

The present invention also provides a method for inducing hypermyelination of a neurite in nervous tissue. The hypermyelination of the neurite may take place in the nerves of both the CNS and the PNS. Hypermyelination of neurites may be measured or detected by known procedures, including Western blotting for myelin-specific and axon-specific proteins, electron microscopy in conjunction with morphometry, and any of the methods, molecular procedures, and assays disclosed herein. As used herein, the term “inducing hypermyelination of a neurite” means activating, inducing, or stimulating growth or regrowth of the myelin of a neurite that has degenerated, wherein the amount or extent of myelination is greater than that which would be expected in a normal or healthy neurite. As further used herein, the term “hypermyelination” refers to a g-ratio greater than 0.6.

The g-ratio is one measure of the integrity of the axon:myelin association. Specifically, the g-ratio is defined as the axonal diameter divided by the total diameter of the axon and myelin. This ratio provides a reliable measure of relative myelination for an axon of any given size (Bieri et al., Abnormal nerve conduction studies in mice expressing a mutant form of the POU transcription factor, SCIP. J. Neurosci. Res., 50:821-28, 1997). Numerous studies have documented that a g-ratio of 0.6 is normal for most fibers (Waxman and Bennett, Relative conduction velocities of small myelinated and nonmyelinated fibres in the central nervous system. Nature New Biol., 238:217, 1972), and alteration in the g-ratio generally reflects either axonal atrophy or demyelination (Moore et al., Simulations of conduction in uniform myelinated fibers. Relative sensitivity to changes in nodal and internodal parameters. Biophys. J, 21:147-60, 1978).

As disclosed herein, the method of the present invention comprises contacting at least one Schwann cell adjacent to a neurite in nervous tissue with an immunophilin ligand. The nervous tissue may be damaged or healthy/undamaged. However, in one embodiment of the present invention, the nervous tissue comprises damaged peripheral neurons. The amount of immunophilin ligand that is used is an amount effective to induce hypermyelination of a neurite. This amount may be readily determined by the skilled artisan, based upon known procedures, including analysis of in vivo dose curves and measurement of quantities of myelin-specific and axon-specific proteins per unit length of nerve, and methods disclosed herein.

The method of the present invention may be used to induce hypermyelination of a neurite in vitro, ex vivo, or in vivo in a subject, in accordance with methods described above. In one embodiment of the present invention, the immunophilin ligand is FK506 or an FK506 derivative. The FK506 derivative for use in the present invention may be nonimmunosuppressive. In a preferred embodiment of the present invention, the nonimmunosuppressive FK506 derivative is GM-284.

As shown by Bieri et al (Abnormal nerve conduction studies in mice expressing a mutant form of the POU transcription factor, SCIP. J. Neurosci. Res., 50:821-28, 1997), animals with hypermyelination conduct the nerve action potential better and faster than normal animals without hypermyelination. Accordingly, hypermyelination may be a desirable condition for subjects in whom the action potential is conducted more slowly than normal, or for subjects with inherited demyelinating neuropathies. It is also believed that, by inducing remyelination, immunophilin ligands will be useful for the treatment of conditions associated with nervous tissue degeneration. It is further believed that immunophilin ligands, including GM-284, would be effective either alone or in combination with other therapeutic agents that are typically used in the treatment of these conditions. Accordingly, the present invention provides a method for treating nervous tissue degeneration in a subject in need of treatment, comprising contacting at least one Schwann cell adjacent to a neurite in damaged nervous tissue in the subject with an amount of an immunophilin ligand effective to induce remyelination of the neurite, thereby treating the nervous tissue degeneration. Examples of nervous tissue degeneration, including peripheral neuropathies, which may be treated by the method of the present invention are discussed above. In a preferred embodiment of the present invention, the peripheral neuropathy is ALS or a hereditary peripheral neuropathy.

FK506 and its nonimmunosuppressive analogues have recently been shown to promote neurite outgrowth in vitro. However, to date, the molecular and cellular mechanisms underlying this activity have not been established. In the present study, the inventors have demonstrated that the neuritogenic actions of this class of compounds are indirect, and are mediated through Schwann cells. In studies designed to elucidate the molecular mechanisms underlying this biology, the inventors have identified a series of transcription factors in Schwann cells that are upregulated in a temporal cascade by FK506 and its related analogues and derivatives. The upregulated genes, SCIP and Brn-5, are members of the POU family of transcription factors. The inventors recently have demonstrated that these gene products regulate the timing and extent of in vivo myelination, and are associated with maintenance of the myelinating state.

Accordingly, the present invention further provides a method for modulating gene expression in a Schwann cell, by contacting the Schwann cell with an immunophilin ligand. The Schwann cell may be in nervous tissue of the CNS (e.g., where astrocytes are in contact with CNS axons) or PNS. In the method of the present invention, the Schwann cell is contacted with an amount of immunophilin ligand effective to modulate gene expression in the Schwann cell. This amount may be readily determined by the skilled artisan, based upon known procedures, including analysis of in vivo dose curves, measurement of quantities of myelin-specific and axon-specific proteins per unit length of nerve, and electron microscopy in conjunction with morphometry, and methods disclosed herein.

As used herein, the term “modulating gene expression” includes altering gene expression by increasing or upregulating gene expression, or by decreasing or downregulating gene expression. By way of example, the expression of a Schwann cell transcription factor may be modulated by contacting a Schwann cell with an immunophilin ligand. Examples of Schwann cell transcription factors which may be modulated by the method of the present invention include, without limitation, SCIP and Brn-5—both of which are members of the POU family of transcription factors—as well as cytokeratin 19, fibrillin 2 (fbn2), IFNγ inducible p58 inhibitor, CD 14, estradiol dehydrogenase, PETA-3, fas-associated factor 1, insulin-like growth factor II, tetraspan TM4SF, hyaluronan-binding protein/HGF activator, integrin β-2, carbonic anhydrase 1, UNC-51-like kinase (ULK) 2, EXO70 protein (Exo70), and neuropilin. Modulation of gene expression may be measured or detected by known procedures, including cDNA-array assays of gene expression and any of the methods, molecular procedures, and assays disclosed herein.

The method of the present invention may be used to modulate gene expression in a Schwann cell in vitro, ex vivo, or in vivo in a subject, in accordance with methods described above. In one embodiment of the present invention, the immunophilin ligand is FK506 or an FK506 derivative. The FK506 derivative for use in the present invention may be nonimmunosuppressive. In a preferred embodiment of the present invention, the nonimmunosuppressive FK506 derivative is GM-284.

It is believed that, by modulating gene expression in Schwann cells, immunophilin ligands will be useful for the treatment of conditions associated with nervous tissue degeneration. It is further believed that immunophilin ligands, including GM-284, would be effective either alone or in combination with other therapeutic agents that are typically used in the treatment of these conditions. Accordingly, the present invention provides a method for treating nervous tissue degeneration in a subject in need of treatment, comprising contacting at least one Schwann cell adjacent to a neurite in damaged nervous tissue in the subject with an amount of an immunophilin ligand effective to modulate gene expression in a Schwann cell, thereby treating the nervous tissue degeneration. Examples of nervous tissue degeneration, including peripheral neuropathies, which may be treated by the method of the present invention are discussed above. In a preferred embodiment of the present invention, the peripheral neuropathy is ALS or a hereditary peripheral neuropathy.

The present invention also provides a method for treating a peripheral neuropathy in a subject in need of treatment, by modulating expression of the Schwann cell transcription factors, SCIP or Brn-5, in the subject. In accordance with methods described above, the peripheral neuropathy in the subject may be treated, and expression of SCIP or Brn-5 may be modulated, by enhancing regeneration of at least one neurite in the subject, by enhancing remyelination of at least one neurite in the subject, and/or by inducing hypermyelination of a neurite in nervous tissue of the subject. Examples of peripheral neuropathies that may be treated by the method of the present invention are discussed above. In a preferred embodiment of the present invention, the peripheral neuropathy is ALS or a hereditary peripheral neuropathy.

The method of the present invention may be used to treat a peripheral neuropathy in vivo in a subject by administering an immunophilin ligand to the subject, as described above. In one embodiment of the present invention, the immunophilin ligand is FK506 or an FK506 derivative. The FK506 derivative for use in the present invention may be nonimmunosuppressive. In a preferred embodiment of the present invention, the nonimmunosuppressive FK506 derivative is GM-284.

The immunophilin ligand of the present invention is administered to a subject in need of treatment for a peripheral neuropathy in an amount that is effective to treat the nervous tissue degeneration in the subject. As used herein, the phrase “effective to treat nervous tissue degeneration” means effective to ameliorate or minimize the clinical impairment or symptoms of the nervous tissue degeneration. For example, where the nervous tissue degeneration is a peripheral neuropathy, the clinical impairment or symptoms of the peripheral neuropathy may be ameliorated or minimized by alleviating vasomotor symptoms, increasing deep tendon reflexes, reducing muscle atrophy, restoring sensory function, and strengthening muscles. The amount of immunophilin ligand effective to treat nervous tissue degeneration in a subject in need of treatment therefor will vary depending upon the particular factors of each case, including the type of nervous tissue degeneration, the stage of the nervous tissue degeneration, the subject's weight, the severity of the subject's condition, and the method of administration. This amount may be readily determined by the skilled artisan, based upon known procedures, including clinical trials, and methods disclosed herein.

The present invention also provides a method for treating a peripheral neuropathy in a subject in need of treatment, by administering to the subject an amount of GM-284 effective to treat the peripheral neuropathy in the subject. Examples of peripheral neuropathies that may be treated by the method of the present invention are discussed above. In a preferred embodiment of the present invention, the peripheral neuropathy is ALS or a hereditary peripheral neuropathy. The method of the present invention may be used to treat a peripheral neuropathy in vivo in a subject by administering GM-284 to the subject in an amount that is effective to treat the peripheral neuropathy in the subject, as defined above.

The present invention is described in the following Examples, which are set forth to aid in the understanding of the invention, and should not be construed to limit in any way the scope of the invention as defined in the claims which follow thereafter.

### EXAMPLES

### Example 1

### Antibodies, Growth Factors, and Reagents

The following commercially available antisera were purchased from their respective vendors: anti-phospho-T202/Y204 MAP kinase monoclonal antibody (New England Biolabs, #9106S), anti-NGF IgG-1 (Boehringer Mannheim, #1087-754), and anti-Thy 1.1 monoclonal antibody (ATCC, clone TIB-100). 2.5S nerve growth factor (NGF) was purchased from Harlan Bioproducts for Science (#BT-5017), reconstituted in sterile PBS containing 1% BSA, and stored frozen in aliquots at −70° C. without freeze/thawing. FK506 and ³H-FK506 (37 Mbq/ml) were purchased from CalBiochem, La Jolla, Calif. (#342500) and NEN Life Sciences, Boston, Mass. (NET1095), respectively. Tissue-culture-grade brain pituitary extract and forskolin were purchased from Sigma Chemicals, St. Louis, Mo., and N2 supplement was obtained from Gibco-BRL, Gaithersburg, Md.

### Example 2

### Tryptophan Fluorescence Measurements

The fluorescent measurements were based predominantly on the interactions of the immunophilin ligands with aromatic amino acids, particularly tryptophan 89 and FKBP52 (Rouviere et al., Immunosuppressor binding to the immunophilin FKBP59 affects the local structural dynamics of a surface beta-strand: time-resolved fluorescence study. Biochemistry, 36:7339-52, 1997). Measurements were made with a Perkin-Elmer 760-40 fluorescence spectrophotometer, at an excitation wavelength of 290 nm (slit width of 2 nm) and an emission wavelength of 345 nm (slit width of 6 nm). A circulating water bath was used to maintain the sample temperature at 18° C., and a mini magnetic stirrer was used to mix the solution (0.3 mM, FKBP52 final concentration) in a 2.5-ml quartz fluorescence cuvette cell.

To obtain titration curves, immunophilin ligands from a stock solution of 0.25 mM were added at 0.2-0.5 μl increments, to achieve a final concentration of 4.0 μM in phosphate-buffered saline (PBS) containing 1 mM DTT. The change in protein concentration resulting from addition of FK506 or GM-284 (from 0.02 μM to 4.0 μM, over a series of 17 independent measurements) was properly corrected for in the final calculations. The experimental data was fitted to the following equation: F=Fₘₐₓ×[immunophilin ligand]/(k_{d}+[immunophilin ligand]), where F is the measured protein fluorescence intensity at each ligand concentration, Fₘₐₓ is the maximal observed fluorescence intensity of FKBP52 when saturated with ligand, and [immunophilin ligand] is the final peptide concentration at each data point. Non-linear regression curves were fitted using SigmaPlot and the following equation: F(x)=a+(X×b)/(Y+c), where X is the free ligand concentration, Y is the concentration of FKBP52, and c is the k_{d}.

### Example 3

### ³H-FK506 Competition Assay

Competition of ³H-506 binding to FKBP52 was carried out on GSH Sepharose beads from GST-FKBP52 expressed in bacteria. Briefly, GST-FKBP52 was constructed by PCR amplification of full-length FKBP52 from the pcDNA3 vector (Invitrogen, Carlsbad, Calif.), with FKBP52-specific primers flanked by EcORI/BamH1 restriction endonuclease sites. A 1.4-kb fragment was subsequently ligated into EcORI/BamH1, and digested with CIP-treated pGEX-6p to obtain a GST-fusion protein. The correct reading frame was verified by DNA sequencing. GST-FKBP52 fusion proteins were generated upon induction with 0.1 mM IPTG (from a 500-ml culture), and bacterial lysates were incubated with GSH Sepharose beads (Pharmacia, Peapack, N.J.) to collect FKBP52. Following several washes, first in bacterial lysis buffer and subsequently in PBS, the FKBP52 was over 90% pure, and was used for in vitro competition studies.

### Example 4

### DRG Isolation and Neuronal Culture

Explanted DRG ganglia were dissected from P1 to P3 C57B1/6 mice, as previously described (Weinstein et al., Targeted expression of an oncogenic adaptor protein v-Crk potentiates axonal growth in dorsal root ganglia and motor neurons in vivo. Brain Res. Dev. Brain Res., 116:29-39, 1999). Briefly, DRGs were dissected and placed in ice-cold PBS containing 2% glucose, then transferred onto polyornithine/laminin-coated tissue culture plates (Biocoat, Becton Dickinson Laboratories) in DMEM-plating medium overnight. The plating medium consisted of Dulbecco's minimal essential plating medium (DMEM) supplemented with 10% fetal calf serum (FCS; Hyclone), 0.6% glucose, 2 mM glutamine, 100 U/ml penicillin, 100 μg/ml streptomycin, and 20 mM HEPES. Following overnight incubation, the medium was carefully removed and replaced with DMEM, as above, except that the DMEM contained 1% FCS and N2 supplement, and either 100 ng/ml 2.5S NGF or 1 μg of an immunophilin ligand (either FK506 or GM-284, unless otherwise indicated). For experiments employing anti-NGF neutralizing antibodies, cultures were incubated with 0.25 μg/ml anti-NGF antiserum, and replaced every two days. Cultures were kept at 37° C., in 5% CO₂, for up to 1 week, at which time the extent of well-defined neurite outgrowth was assessed.

To generate neuron-enriched DRG cultures, DRGs were isolated from embryonic (E18) C57B1/6 mice, and processed in dissection media containing Hank's CMF saline, with 0.05% collagenase and 0.25% trypsin, for 30 min at 37° C. Neurons were obtained by trituration of ganglia with fire-polished Pasteur pipettes of decreasing diameter. Thereafter, the cellular suspension was washed twice in Dulbecco's modified Eagle medium supplemented with 10% fetal calf serum and 2% glucose. Freshly triturated neurons were incubated in 10% FCS-DMEM supplemented with 5-fluorouracil (Sigma, F-0503) and uridine (Sigma, U3003) for 48 h to remove non-neuronal proliferating cells, in the presence of either 100 ng/ml NGF or 1 μM GM-284. After 48 h, cultures were continued with these agents, but without mitotic inhibitors, for an additional 6 days.

### Example 5

### PC12 Cell Culture and Schwann Cell/PC12 Co-cultures

Wild-type and TrkA-overexpressing PC12 cells (Hempstead et al., Overexpression of the trk tyrosine kinase rapidly accelerates nerve growth factor-induced differentiation. Neuron, 9:883-96, 1992) were maintained in DMEM supplemented with 10% calf serum and 5% horse serum, the latter of which was also supplemented with 200 μg/ml G418 to maintain TrkA selection. Cultures were treated with 100 ng/ml NGF or the immunophilin ligands, or treated with Schwann-cell-conditioned medium supplemented with 5% horse serum, as indicated below. To generate GFP-expressing PC12 cells, a bicistronic pCX-bsr retroviral vector (Escalante et al., Phosphorylation of c-Crk II on the negative regulatory Tyr222 mediates nerve growth factor-induced cell spreading and morphogenesis. J. Biol. Chem., 275:24787-797, 2000), which drives expression of green fluorescent protein (GFP), was used to infect dividing PC12 cells.

GFP-expressing virus was produced by lipofectamine-mediated transfection of 1.0 μg of pCX-bsr DNA, together with 1.0 μg of pC-Eco retroviral DNA, into the Bosc23 replication-incompetent ecotropic packaging line (Retromax™, Imgen), according to the manufacturer's protocols. The transfection medium was replaced with 10 ml of DMEM containing 10% fetal calf serum, for 24 h, and then collected and frozen at −70° C. The cells were re-fed with fresh DMEM (10 ml), and maintained for an additional 16 h. A total of 20 ml of virus-containing tissue-culture media were pooled, centrifuged at 15,000×g for 3 h, and resuspended into 2.0 ml of PCl₂ cell medium containing 7 μg/ml polybrene. PC12 cells, seeded at 50% confluency, were infected with the concentrated stock for 48 h. The medium was then changed, and the cells were incubated for an additional 48 h. Between 20 and 30% of PC12 cells were GFP positive, and expression was detectable for up to one week.

To generate primary Schwann cells, neonatal Sprague-Dawley rats were sacrificed by decapitation, and 2-cm segments (approximately) of sciatic nerve were excised and placed in ice-cold PBS. Following removal of the epineurium, the nerves were pooled and placed in Hank's balanced salt solution containing 0.1% trypsin for 30 min at 37° C. Subsequently, partially-dissociated cells were plated onto polylysine/laminin-coated tissue culture plates, and enriched Schwann cells were further purified by the activation of anti-Thy 1.1 monoclonal antibodies and complement, in order to lyse proliferating fibroblasts (Wu and Weinstein, “Isolation and Purification of Primary Schwann Cells”, in Current Protocols in Neuroscience, Crawley et al., eds. (New York, N.Y.: John Wiley & Sons, 1999). The Schwann cells were maintained in DMEM containing 10% FCS supplemented with 50 ng/ml GGF and 2 μg/ml forskolin. However, these latter compounds were removed 48 h prior to the addition of immunophilin ligands. For the Schwann cell/PC12 co-cultures, monolayers of Schwann cells were prepared and overlaid with pCX-expressing PC12 cells, and cultures were incubated in the presence of either 100 ng/ml NGF or 1 μg/ml GM-284 for 48 h.

### Example 6

### Immunofluorescence Microscopy

Primary Schwann cells were seeded onto Poly-D-lysine-coated glass chamber slides, in serum-free medium, and incubated overnight in a 37° C. incubator. On the following day, the cells were gently washed once with PBS, and fixed by incubating in 4% paraformaldehyde in PBS, for 30 min at room temperature. After fixing, the cells were blocked in PBS with 10% goat serum, for 1 h at room temperature. For permeabilization, 0.1% Triton X-100 was added to the blocking buffer. The cells were then washed 3 times in PBS, and incubated with primary antibody—either rabbit anti-S100β (Dako Corporation, Carpinteria, Calif.) or rabbit anti-FKBP52 (StressGen Biotechnologies Corporation, Inc., Victoria, BC, Canada)—at 1:1000, for 1 h at room temperature. Cells were then washed 3 times in PBS, and incubated with 1:200 dilution of FITC-conjugated goat anti-rabbit IgG (Jackson ImmunOResearch Laboratories, Inc., West Grove, Pa.), for 1 h at room temperature in the dark. Following secondary-antibody staining, cells were washed 3 times and incubated with Hoechst stain (Molecular Probes, Eugene, Oreg.), diluted 1:1000, for 5 min at room temperature. Cells were viewed on a Nikon Eclipse TE 300 microscope equipped with an epifluorescence filter, and photographs were taken using a cooled CCD camera.

### Example 7

### Electron Microscopy

Mice were anesthetized (i.p.) with 0.5 cc of 2.5% Avertin/saline, before perfusion via the left ventricle. The animals were perfused for 30 min at 37° C., with a rat Ringer's solution containing heparin (2.0 ml/L; stock=10,000 units/ml) and 2% lidocaine (3.0 ml/L), followed by perfusion for 7-10 min with 2% glutaraldehyde/1% paraformaldehyde in 0.15 M sodium cacodylate, pH 7.2. Approximately 1 cm of sciatic nerve was dissected from the hind leg (from the hip to the knee), and split into 3 sections (proximal, medial, and distal). A 2-mm length was taken from each of the three sections, then immersion fixed. The primary fixation was carried out for 2-4 h total, at 4° C., with 2% glutaraldehyde/1% paraformaldehyde in 0.15 M sodium cacodylate buffer, pH 7.2. Tissues were rinsed 6 times, for 20 min each, then rinsed overnight at 4° C. in 0.15 M sodium cacodylate buffer, pH 7.2.

Secondary fixation was carried out for 4 h at 4° C. in 1% osmium-tetroxide/1.5% potassium-ferrocyanide in 0.15 M sodium cacodylate buffer, pH 7.2. Tissues were rinsed 3 times, for 10 min each, in Millipore-filtered water at 4° C., then stained en bloc in 2% uranyl acetate (aq) at 4° C. for 1 h. At this point, the tissues were dehydrated once for 8 min in a graded ethanol series starting with water, 30%, 50%, 70%, 95%, and then dehydrated twice, for 10 min each, in 100% ethanol and then in propylene oxide. After dehydration, the nerve tissue was infiltrated with propylene oxide/Durcupan (Fluka Chemika-BioChemika, Ronkonkoma, N.Y.), in a 25/75 ratio, for 60 min at room temperature. This was followed by infiltration three times, for 120 min each, in Durcupan resin at room temperature. Sciatic nerves were flat-embedded in fresh Durcupan resin, and polymerized for 24-36 h at 65° C. 1-μM-thick sections were stained with Toluidine blue. Silver sections were cut on a Diatome diamond knife, and stained with 2% uranyl acetate for 30 min at room temperature, and with Reynold's lead citrate for 7 min. Thin sections were viewed at 60 kv, and photographed on a JEOL 100 CX conventional transmission electron microscope.

### Example 8

### Cell Lysis and Western Blotting

293T cells were lysed in ice-cold HNTG buffer (20 mM HEPES, pH 7.5; 150 mM NaCl; 10% glycerol; and 1% Triton X-100) containing 0.1 mM sodium molybdate, 1 mM sodium vanadate, 1 mM phenylmethylsulfonyl fluoride (PMSF), and 10 μg/ml aprotinin. After 10 min at 4° C., cleared lysates were subjected to SDS-PAGE and Western blot analysis using standard protocols. Blots were incubated with the indicated primary antibodies and the appropriate horseradish-peroxide-(HRP)-conjugated secondary antibodies, followed by detection using enhanced chemiluminescence (ECL) reagent (Renaissance, NEN).

### Example 9

### RNA Blot Analysis

20 μg of total RNA was prepared as previously described (Wu et al., The POU gene brn-5 is induced by neuregulin and is restricted to myelinating Schwann cells. Mol. Cell Neurosci., 17:683-95, 2001). Thereafter, total RNA was separated on 1% MOPS-formaldehyde agarose gels, transferred to MAGNA nylon transfer membrane (MSI), UV cross-linked (120 mJ/cm 2) using a UV crosslinker (FB-UVXL-1000, Fisher Biotech), and hybridized with ³²P-dCTP random-labeled (RTS RadPrime DNA Labeling System, Gibco BRL), probes purified by Sephadex G-50 column (Pharmacia, Peapack, N.J.).

The Brn-5 probe contains the rat Brn-5 coding region (generated by PCR), and was kindly provided by Dr. Bogi Andersen at the University of California at San Diego. This probe is 93% identical to the mouse Brn-5 mRNA. The SCIP probe was a 1.10-kb SmaI cDNA fragment from mouse SCIP DNA. The cyclophilin probe was identical to that previously described (Hasel and Sutcliffe, Nucleotide sequence of a cDNA coding for mouse cyclophilin. Nucleic Acids Res., 18:4019, 1990). The cyclophilin probe and 18S probes (Ambion) were used as controls for gel-loading differences. The membranes were prehybridized for 4 h at 42° C. in hybridization buffer, and hybridized for 16-18 h in 50% formamide, 5×SSCP, 2× Denhart's, 0.1% SDS, and 200 μg/ml ssDNA. After prehybridization and hybridization, the filters were washed 3 times, for 15 min each, in 2×SSC/1% SDS; the filters were then washed 2 times, for 10 min each, in 0.2×SSC/0.5% at 65° C.

### Example 10

### Sciatic Nerve Crush and Treatment with GM-284

Deep anesthesia was obtained with i.p. injection of Avertin. Electrophysiology recordings were obtained, as previously described (Bieri et al., Abnormal nerve conduction studies in mice expressing a mutant form of the POU transcription factor SCIP. J. Neurosci. Res., 50:821-28, 1997). Immediately afterward, the right sciatic nerve of each animal was exposed and crushed 2 times, for 30 sec each, using a #5 Dumont forceps. In brief, the skin was prepped bilaterally prior to electrophysiologic testing. Prior to surgery, the area was cleaned, and a hockey-stick incision was made over the right lower quadrant, from the sciatic notch to the knee. The overlaying muscle was bluntly dissected, and the sciatic nerve was exposed and crushed. Following crush, the muscle was closed with 1-2 sutures of 40 gut, and the skin was closed with 40 braided silk and/or staples. The animal was placed in the left lateral position, and allowed to recover in its cage. All procedures were uneventful, and well tolerated by the animals. All animals were alert within 2 h of the procedure.

Summarized below are results obtained by the inventors in connection with the experiments of the above Examples:

NGF and GM-284 Promote Neurite Outgrowth From Dorsal Root Ganglia Explants.

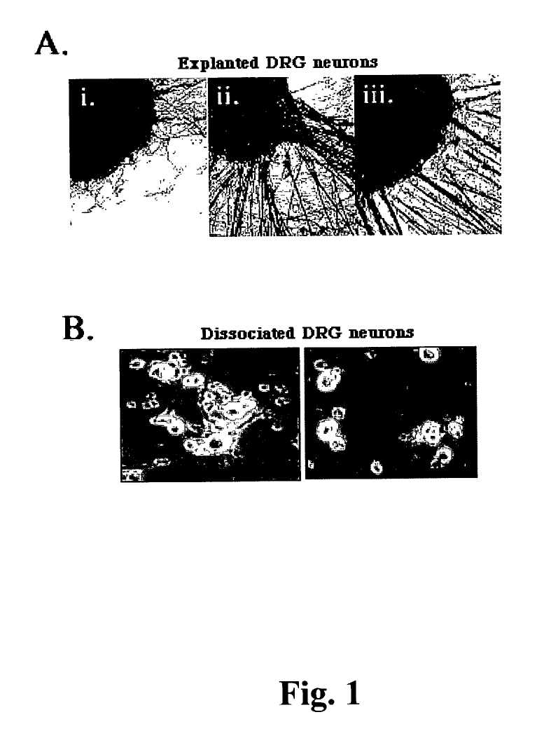

DRG neurons are bipolar cells in vivo, sending one axon to peripheral targets, and a second axon into the spinal cord, where the DRG neurons contribute to the ascending dorsal tracts. A major subset of DRG neurons are NGF-responsive; these can be rescued from apoptosis in vitro when exposed to saturating concentrations of this neurotrophin (Weinstein et al., Targeted expression of an oncogenic adaptor protein v-Crk potentiates axonal growth in dorsal root ganglia and motor neurons in vivo. Brain Res. Dev. Brain Res., 116:29-39, 1999). In addition to survival, NGF induces robust neurite outgrowth when whole DRGs are explanted and cultured in the presence of this neurotrophin (cf panels i and ii in FIG. 1A). In addition to the neurotrophins, other classes of molecules have been demonstrated to promote neurite outgrowth from DRG neurons. One such class of compounds comprises the immunophilin ligands. The inventors have been interested in the actions of a member of this growing family of drugs (namely, a novel, nonimmunosuppressive mimetic of FK506, referred to herein as GM-284), and its ability to influence neuritogenesis. As shown in FIG. 1, GM-284 appears to be as active as NGF in inducing neuritogenesis in DRG explants. (The formal assignment of GM-284 to the group of nonimmunosuppressive immunophilin ligands is demonstrated in detail below.)

DRG explants are complex tissues, containing both Schwann cells and neurons. Accordingly, it is possible that GM-284 acts to promote neurite outgrowth directly, by acting on neurons, or indirectly, by acting on adjacent Schwann cells, which then act to promote neuritogenesis. To differentiate these possibilities, sensory neurons from DRG were purified as described (Wood et al., Studies of the initiation of myelination by Schwann cells. Ann. NY Acad. Sci., 605:1-14, 1990), and contaminating Schwann cells were removed by treatment of the cultures with anti-mitotic drugs. Like two other nonimmunosuppressive immunophilin ligands, GPI-1046 and V10,367, GM-284 is unable to mediate neurite outgrowth in the absence of other signaling molecules. As shown in FIG. 1B, for example, purified sensory neurons were treated with NGF as a positive control, and sister cultures were treated with GM-284 alone. In the sister cultures, there was a complete failure to elaborate processes, thereby demonstrating that, like the other immunophilin ligands, GM-284 does not act directly on neurons in promoting axonogenesis.

GM-284-Induced Neuritogenesis Lies Outside of the Neurotrophin-Signaling Pathway.

The above data suggest that GM-284 activity likely acts on Schwann cells, and that these cells, in turn, act on neurons to promote neurite growth. Schwann cells are known to make a number of neurotrophins, including NGF (Rogister et al., Transforming growth factor beta as a neuronoglial signal during peripheral nervous system response to injury. J. Neurosci. Res., 34:32-43, 1993), BDNF (Friedman et al., Trophic factors in neuron-Schwann cell interactions. Ann. N.Y. Acad. Sci., 883:427-38, 1999), NT-3, and GDNF (Wiklund et al., Mitogen-activated protein kinase inhibition reveals differences in signaling pathways activated by neurotrophin-3 and other growth-stimulating conditions of adult mouse dorsal root ganglia neurons. J. Neurosci. Res., 67:62-68, 2002). Each of these neurotrophins acts on differing subsets of DRG neurons, although the vast majority of DRG neurons are NGF-responsive.

Signaling by the neurotrophins, while mediated by different cell-surface receptors, converges on the extensively studied and well-characterized MAP kinase pathway. The number and extent of neurites produced in the GM-284-treated DRGs explants suggested that NGF or NGF-like activity was involved. Therefore, the inventors explored the possibility that GM-284 might signal through NGF directly, or through the MAP kinase cascade. To investigate this possibility, sister cultures were established in the presence of NGF or GM-284, and pretreated with either function-blocking anti-NGF, or the MAP kinase (MEK1)-specific inhibitor, PD 098059 (Alessi et al., PD 098059 is a specific inhibitor of the activation of mitogen-activated protein kinase in vitro and in vivo. J. Biol. Chem., 270:27489-494, 1995).

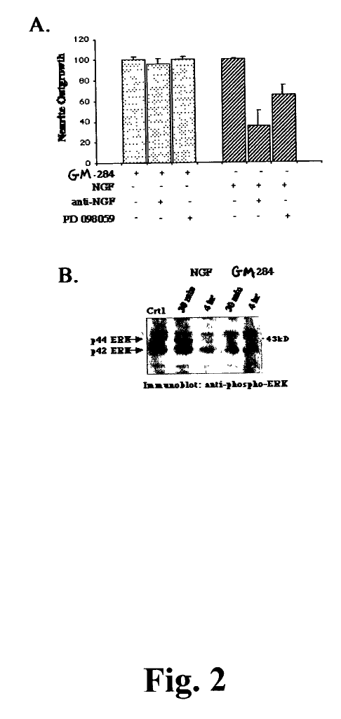

PD 098059 inhibits MAP kinase activation that is a characteristic of NGF-induced TrkA binding (Gomez and Cohen, Dissection of the protein kinase cascade by which nerve growth factor activates MAP kinases. Nature, 353:170-73, 1991; McMahon et al., Expression and coexpression of Trk receptors in subpopulations of adult primary sensory neurons projecting to identified peripheral targets. Neuron, 12:1161-171, 1994), as well as activation induced by binding of other neurotrophin receptors (Wiklund et al., Mitogen-activated protein kinase inhibition reveals differences in signaling pathways activated by neurotrophin-3 and other growth-stimulating conditions of adult mouse dorsal root ganglia neurons. J. Neurosci. Res., 67:62-68, 2002). Consistent with that which has been shown by many other groups, NGF-neutralizing antibodies and MEK inhibition significantly decreased NGF-dependent neurite outgrowth (by approximately 60% and 40%, respectively). In contrast, these inhibitors had no effect on GM-284-mediated neurite outgrowth (FIG. 2B). Furthermore, GM-284 showed no activity on Erk phosphorylation in DRG neurons, whereas NGF clearly activated Erk (FIG. 2C). Taken together, these data suggest that GM-284 acts through a non-TrkA/Ras/Erk-dependent signaling pathway that differs from the known neurotrophin-activation pathways.

GM-284 is an Immunophilin Ligand that Binds to FKBP52.

The nonimmunosuppressive immunophilin ligands, by definition, retain the ability to bind one or more immunophilins, but fail to inhibit calcineurin; thus, they are devoid of immunomodulatory activity. Several such compounds have been produced by the inventors. Based upon the robust neuritogenic activity of one of these compounds, GM-284 (as demonstrated in FIG. 1), the inventors reasoned that identification of the intracellular GM-284 ligand(s) might give insight into the mechanism(s) of action leading to neurite promotion. Moreover, data shown in FIG. 1 suggest that GM-284 acts indirectly, through the Schwann cell.

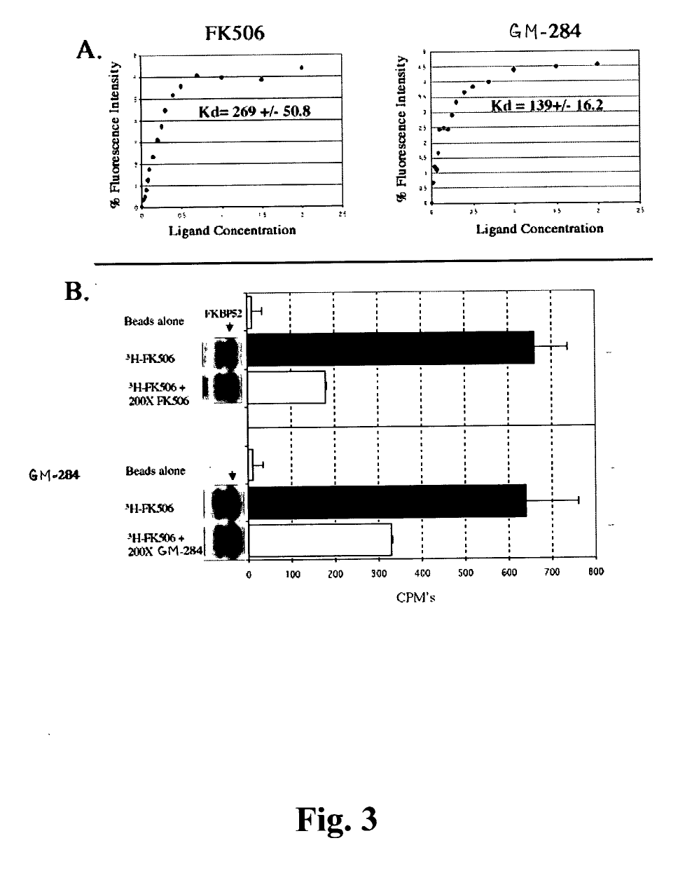

One candidate receptor for GM-284 is FKBP52, a 56-kDa protein that been shown to bind other immunophilin ligands (Peattie et al., Expression and characterization of human FKBP52, an immunophilin that associates with the 90-kDa heat shock protein and is a component of steroid receptor complexes. Proc. Natl. Acad. Sci. USA, 89:10974-978, 1992), and which has been implicated in mediation of the neuritogenic effects of FK506 (Gold, B. G., FK506 and the role of the immunophilin FKBP-52 in nerve regeneration. Drug Metab. Rev., 31:649-63, 1999). As shown in FIG. 3A, Schwann cells which were isolated and purified to homogeneity as described (Wu and Weinstein, Isolation and Purification of Primary Schwann Cells (New York: John Wiley & Sons, 1999) express FKBP52, further supporting the possible role of Schwann cells as mediators of GM-284 signaling.

To determine if FKBP52 was a receptor for GM-284, the inventors took advantage of a recently reported biophysical technique, quantitative tryptophan fluorescence spectroscopy (QTFS), which allows for the accurate determination of binding affinities between any Trp-containing molecule and its ligand. Previously reported studies by Rouviere et al. have shown that one of the two Trp residues identified in FKBP52 (Trp-89), which is conserved in virtually all known immunophilins, is buried in the hydrophobic core of the molecule, and participates in ligand binding (Rouviere et al., Immunosuppressor binding to the immunophilin FKBP59 affects the local structural dynamics of a surface beta-strand: time-resolved fluorescence study. Biochemistry, 36:7339-352, 1997).

To determine if QTFS was sensitive enough to demonstrate interactions of GM-284 and FKBP52, the inventors conducted pilot QTFS studies to measure the interaction between FK506 and the inventors' recombinant FKBP52. As shown in FIG. 3B (left panel), the FK506-FKBP52 dissociation constant (k_{d}) of 269 nm±50.8 closely agrees with the values reported by Rouviere et al. (k_{d}=202 nm±9). Measurements of GM-284-FKBP52 interactions (FIG. 3B, right panel) by QTFS demonstrated a k_{d} of 139 nm+16.2, raising the possibility that GM-284 and FK506 may interact with FKBP52 via a common or related mechanism. To test this, the inventors established competition assays in which excesses of cold FK506 or GM-284 were used to compete for binding of ³H-FK506 bead-immobilized FKBP52. The inventors' data demonstrate that GM-284 is an immunophilin ligand, and that the parent molecule, FK506, and the FK506-mimetic molecule, GM-284, bind to FKBP52 with similar affinities (FIG. 3C).

Neuron/Schwann Cell Co-Cultures Rescue GM-284-Mediated Neurite Formation.

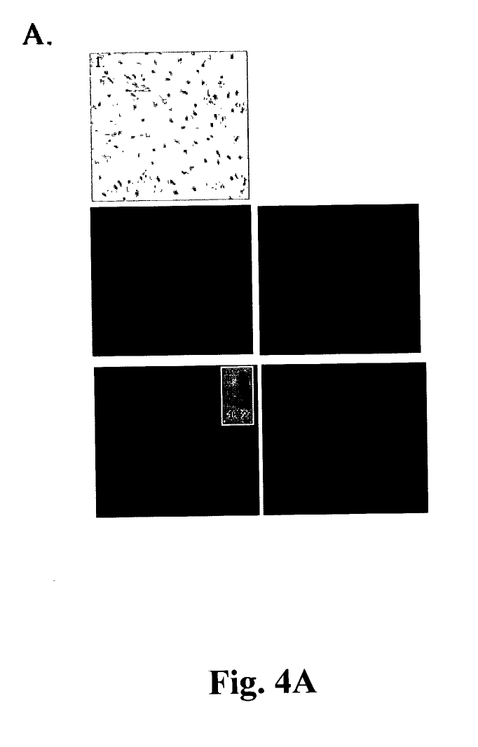

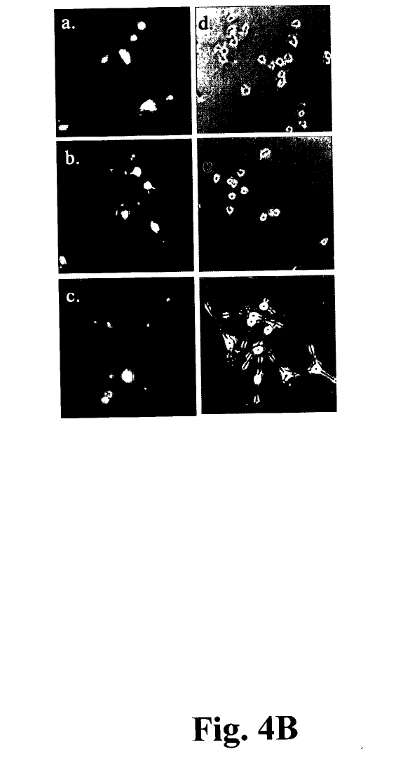

To more directly examine the involvement of Schwann cells in mediating the neuritogenic capacities of GM-284, PC12 cells were infected with GFP-expressing retrovirus, and co-cultured on monolayers of Schwann cells, in the presence of either NGF, GM-284, or vehicle, for 48 h (FIG. 4B, panels a-c). The results indicate that PC12 cells co-cultured with Schwann cells, in the presence of GM-284, undergo extensive neuritogenesis that is comparable to axon growth in the presence of Schwann cells and NGF. To test further whether GM-284 alters expression of one or more Schwann cell surface molecules, or acts to induce one or more secreted factors that promote axonogenesis, purified cultures of Schwann cells were treated with either 1 μM GM-284 or vehicle for 48 h, and the resulting supernatants (“conditioned media”, CM) were added to naïve TrkA-overexpressing PC12 cells for an additional 72 h (FIG. 4B, panels d-e). CM from naïve Schwann cells were inactive in inducing neurite outgrowth (panel d), whereas CM from GM-284-treated Schwann cells promoted significant neurite outgrowth that was as robust as that which was seen when the neuronal cells are treated with NGF (cf panels c and i). These data demonstrate that GM-284 mediates a Schwann cell cascade, probably by binding to Schwann cell FKBP52 and thereby triggering one or more events that result in neuritogenesis.

GM-284 Induces Transcriptional Activation in Schwann Cells.

The observations outlined above suggested to the inventors that GM-284 alters gene expression and/or induces post-translational events in the Schwann cell. To test the former hypothesis, the inventors starved Schwann cells of growth factor for 48 h, then treated the cells for either 4 h or 48 h with a 1-μM dose of GM-284. RNA was harvested from the treated cells and from control (vehicle-treated) Schwann cells, and then prepared for cDNA-array analysis. Experimental details of the preparation of the RNA, and the methods for conducting the hybridization, washing, and scanning, are posted at the following http website: //sequence.aecom.yu.edu/bioinf/funcgenomic.html. The results of these analyses are shown in Table 1. In particular, after 4 h of GM-284 treatment, there was a discreet set of 26 genes that was upregulated. In contrast, after 48 h of treatment, a distinct, non-overlapping set of 109 genes was upregulated. Not surprisingly, among the latter set of genes were molecules associated with signaling, including IGF2 and Fas-associated factor 1, as well as genes that have products associated with either cell-cell or cell-matrix interactions, including integrin β-2 and PETA3. These data show conclusively that GM-284 acts as a transcriptional regulator in Schwann cells.

TABLE 1
GM-284 induces Schwann cell gene expression.

| Analysis of | | |
|---|---|---|
| Gene Expression | 4 hours | 48 hours |
| Total upregulated | 26 | 109 |
| genes | | |
| ESTs | 15 | 77 |
| Known genes | 11 | 32 |
| Selected | Cytokeratin 19 | PETA-3 |
| upregulated genes | Fibrillin 2 (fbn2) | Fas-associated factor 1 |
| | IFNγ inducible | insulin-like growth |
| | p58 inhibitor | factor II |
| | CD14 | tetraspan TM4SF |
| | Estradiol dehydrogenase | hyaluronan-binding protein/ |
| | | HGF activator |
| | | Integrin β-2 |
| | | Carbonic anhydrase 1 |
| | | UNC-51-like kinase |
| | | (ULK) 2 |
| | | EXO70 protein (Exo70) |
| | | neuropilin |

As shown in Table 1, Schwann cells were treated with either vehicle or 1 μM GM-284 for either 4 or 48 h. Total RNA was isolated, and prepared for cDNA microarray analysis as described (Peng et al., Microarray analysis of global changes in gene expression during cardiac myocyte differentiation. Physiol. Genomics, 9(3):145-55, 2002). At 4 h, 26 genes were reproducibly upregulated, the majority of which were unknown. At 48 h, a completely separate, non-overlapping set of 109 genes was upregulated, greater than ⅔ of which were ESTs. Taken together, these data demonstrate that GM-284 acts as a transcriptional regulator in Schwann cells.

GM-284 Upregulates Two Members of the POU Family of Transcriptional Regulators.

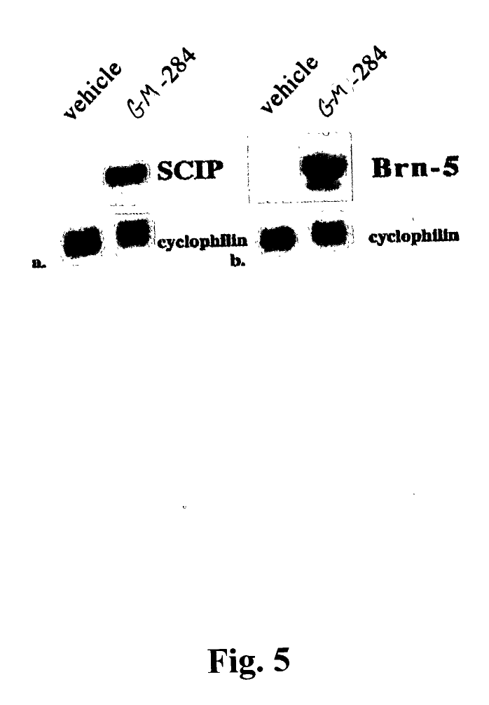

Two of the genes that are upregulated by GM-284 at 48 h, but that are not included in Table 1, are SCIP and Brn-5 (FIG. 5). Previous work by the inventors has shown that expression of the POU transcription factors, SCIP (Oct-6, Tst-1) and Brn-5, correlates with Schwann cell maturation and myelination (Wu et al., The POU gene brn-5 is induced by neuregulin and is restricted to myelinating Schwann cells. Mol. Cell. Neurosci., 17:683-95, 2001; Weinstein et al., Premature Schwann cell differentiation and hypermyelination in mice expressing a targeted antagonist of the POU transcription factor SCIP. Mol. Cell. Neurosci., 6:212-29, 1995). Moreover, the misexpression of one of these genes, SCIP, strongly promotes accelerated rates and extents of axonal regeneration and remyelination (Gondre et al., Accelerated nerve regeneration mediated by Schwann cells expressing a mutant form of the POU protein SCIP. J. Cell. Biol., 141:493-501, 1998).

To verify the cDNA array data, and to determine the level of both Brn-5 and SCIP expression in GM-284-treated Schwann cells, the inventors growth-factor-starved the cells, then treated the cells with GM-284 for 48 h. Thereafter, RNA was isolated and Northern blots were performed for SCIP and Brn-5. As shown in FIG. 5, both SCIP and Brn-5 genes were significantly induced relative to the vehicle-treated cells. These data are in agreement with the cDNA arrays and with the idea that immunophilin ligands regulate a transcriptional program in Schwann cells.

GM-284 Promotes Axonal and Myelin Hypertrophy in the Regenerating Nerve.

The inventors' observations that GM-284 promotes neurite outgrowth and induces the in vitro expression of the POU genes, SCIP and Brn-5, raised the possibility that this compound also might alter either the rate or extent of nerve regeneration following compression. To test this possibility directly, the inventors crushed the right sciatic nerve of adult mice, and randomized the mice into treatment groups (either GM-284 or vehicle), each consisting of eight animals (4 male, 4 female). The treatments were started on the first post-operative day, and were given daily for 34 days.

Prior to surgery, baseline electrophysiological measurements were recorded for each animal. One week following nerve crush, repeated electrophysiological measurements revealed no elicitable responses ipsilateral to the crush, while responses in the contralateral nerve were virtually identical to baseline measurements (Bieri et al., Abnormal nerve conduction studies in mice expressing a mutant form of the POU transcription factor SCIP. J. Neurosci. Res., 50:821-28, 1997; Gondré et al., Accelerated nerve regeneration mediated by Schwann cells expressing a mutant form of the POU protein SCIP. J. Cell. Biol., 141:493-501, 1998), indicating that the surgery caused a complete mechanical transection of the nerve (data not shown). Upon completion of the therapeutic regimen, the animals were sacrificed, and their nerves were processed for electron microscopy, as previously described (Bieri et al., Abnormal nerve conduction studies in mice expressing a mutant form of the POU transcription factor SCIP. J. Neurosci. Res., 50:821-28, 1997).

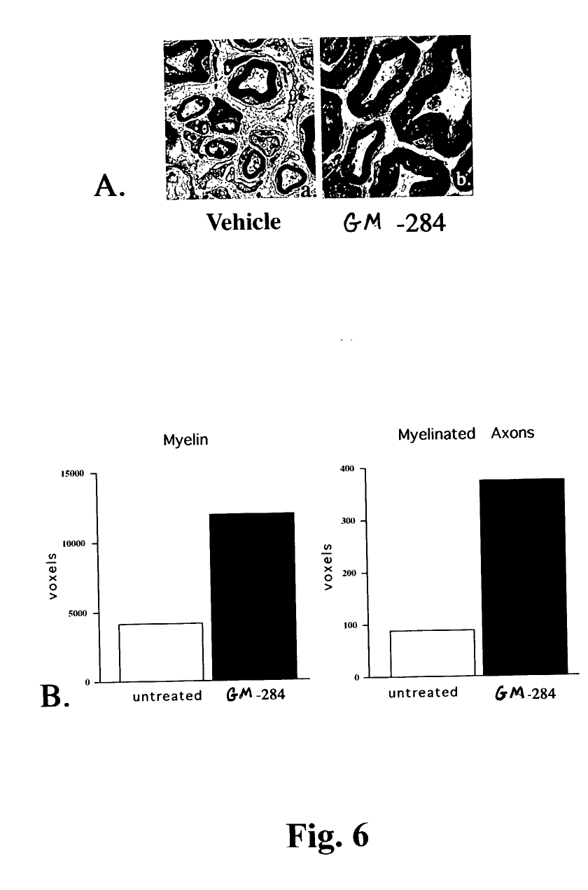

Treatment with GM-284 caused dramatic and numerous changes in the histoarchitecture of the regenerated nerves, irrespective of gender of the animal. As shown in FIG. 6A, GM-284 treatment induced both axonal hypertrophy and a global over-elaboration of the myelin organelle. One measure of the integrity of the axon-myelin association is the g-ratio, which is defined as the axonal diameter divided by the total diameter of the axon-and-myelin unit. This ratio provides a reliable measure of relative myelination for any given size of axon (Waxman and Anderson, Regeneration of spinal electrocyte fibers in Sternarchus albifrons: development of axon-Schwann cell relationships and nodes of Ranvier. Cell & Tissue Res., 208:343-52, 1980). However, in view of the absence of circularity observed in the GM-284-treated nerves, it was virtually impossible to calculate a reliable g-ratio. Rather, the inventors calculated the volumes of both axons and myelin in the GM-284 treatment group and in the vehicle-treated control group using the NIH Image program. In this way, the inventors were able to determine the ratio of axonal volume to myelin volume (volume of axon/volume of myelin) (Gondré et al., Accelerated nerve regeneration mediated by Schwann cells expressing a mutant form of the POU protein SCIP. J. Cell. Biol., 141:493-501, 1998).

Under these conditions, the inventors observed a 20-fold increase in myelin volume, relative to each axon, in the GM-284-treated groups (0.2 vs. 0.01; p<0.0001). In addition, there was an overall increase in size of myelinated axons in the GM-284-treated animals, as compared to the vehicle-treated group, one month following nerve injury (FIG. 6B). These in vivo data correlate very well with the inventors' in vitro results, and they show that GM-284 treatment affects both Schwann cells and their associated axons.

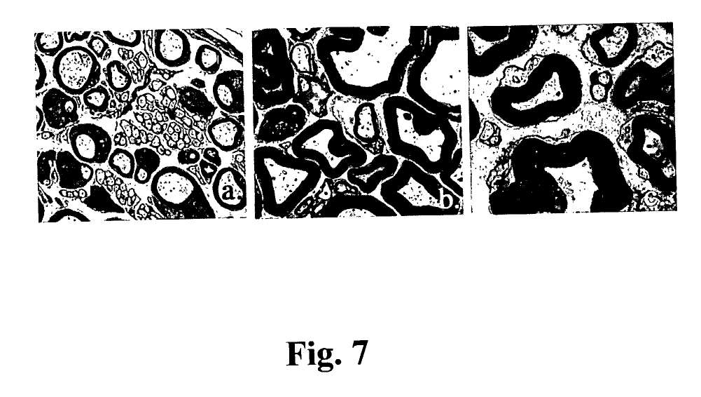

Finally, the histology of the regenerated GM-284-treated nerves was very reminiscent of regenerated nerves in ASCIP mice, which express a dominant-active form of SCIP (Gondre et al., Accelerated nerve regeneration mediated by Schwann cells expressing a mutant form of the POU protein SCIP. J. Cell. Biol., 141:493-501, 1998; Weinstein et al., Premature Schwann cell differentiation and hypermyelination in mice expressing a targeted antagonist of the POU transcription factor SCIP. Mol. Cell. Neurosci., 6:212-29, 1995; Bieri et al., Abnormal nerve conduction studies in mice expressing a mutant form of the POU transcription factor SCIP. J. Neurosci. Res., 50:821-28, 1997). In FIG. 7, the inventors compare the overall appearance of nerves, one month following crushing injury, in animals treated with vehicle (panel a), in ΔSCIP animals (panel b), and in mice treated with GM-284 at 10 mg/kg (panel c). In view of the similarity in appearance of the ΔSCIP- and the GM-284-treated nerves, the inventors' demonstration that the drug upregulates SCIP and Brn-5, the multiple lines of evidence that GM-284 acts to promote neurite outgrowth indirectly through the Schwann cell, and the inventors' previous data that a transactivating SCIP induces axonal and myelin hypertrophy, the inventors posit that GM-284 and other immunophilin ligands initiate a signaling cascade that co-opts and exaggerates the normal biology of Schwann cell/neuron interactions in peripheral nerve regeneration.

Regeneration is a hallmark of the peripheral nervous system (PNS). The similarities between the developing nerve and the regenerating nerve, including axon outgrowth and myelination, have led to the generally accepted view that regeneration recapitulates development. At some levels, this is true: regenerating axons find their targets, and the associated Schwann cells myelinate appropriately sized axons. However, while axons serve as a template to guide Schwann cell migration during development, axons in the regenerating nerve migrate into a milieu in which the Schwann cells are in situ. Moreover, the molecular events which are induced by Schwann cell/axon interactions differ in the regenerating, as compared with the developing nerve (Gondre et al., Accelerated nerve regeneration mediated by Schwann cells expressing a mutant form of the POU protein SCIP. J. Cell. Biol., 141:493-501, 1998; Wu et al., The POU gene bm-5 is induced by neuregulin and is restricted to myelinating Schwann cells. Mol. Cell Neurosci., 17:683-95, 2001).

In both development and regeneration, there is a bi-directional series of forward and reverse signaling events between Schwann cells and axons, which allows for the establishment (development) or re-establishment (regeneration) of the Schwann cell/axon unit that is characteristic of the mature, myelinated nerve in homeostasis. Understanding and exploiting these events will allow for the rational establishment of therapeutic interventions. Herein, the inventors have shown that GM-284 acts on the Schwann cells to modulate the normal events that govern nerve regeneration, thereby augmenting these biologies to enhance normal Schwann cell/axon signals and enhancing regeneration.

The nonimmunosuppressive immunophilin ligands are defined by their ability to bind FKBPs, and their failure to mediate the immune response (Steiner et al., Neurotrophic actions of nonimmunosuppressive analogues of immunosuppressive drugs FK506, rapamycin and cyclosporin A. Nat. Med., 3:421-28, 1997). The observations that these molecules, as well as the parent drug, FK506, enhance peripheral nerve regeneration (Gold, B. G., FK506 and the role of immunophilins in nerve regeneration. Mol. Neurobiol., 15:285-306, 1997; Jost et al., Acceleration of peripheral nerve regeneration following FK506 administration. Restor. Neurol. Neurosci., 17:39-44, 2000) offers significant therapeutic potential.

Following damage in which the nerve sheath is intact, but the axons contained in the sheath are interrupted, peripheral nerves regenerate, albeit very slowly and never quite completely (Griffin and Hoffman, Degeneration and regeneration in the peripheral nervous system. Peripheral Neuropathy, 361-76, 1993). In contrast, interruptions in nerve continuity as a result of trauma or disease profoundly inhibit or diminish regeneration (Strauch et al., The generation of an artificial nerve, and its use as a conduit for regeneration. J. Reconstr. Microsurg., 17:589-98, 2001). Therefore, a compound that can enhance regeneration, while avoiding the immunosuppressive effects of FK506, will fill a significant niche in the treatment of peripheral nerve disease.

Data presented herein demonstrate that GM-284 mediates sensory nerve regeneration in vitro, and that this activity is dependent upon Schwann cells. Specifically, it is shown that GM-284-treated DRG explants have neurite outgrowth which is indistinguishable from NGF-treated sister explants. Moreover, the GM-284-mediated neuritogenesis occurs by a mechanism that does not overlap with neurotrophin-mediated signaling, either at the cell surface or in the intracellular signaling cascade downstream of Trk activation. When the inventors isolated purified cultures of sensory neurons, GM-284 failed to promote axonogenesis, suggesting that the GM-284 neurite-promoting activity in the ganglionic explants acts indirectly on the neurons.

The major constituents of the DRG are neuronal cell soma and Schwann cells, including specialized Schwann cells known as satellite cells. The absence of direct GM-284-mediated activity on neurons, and the cellular makeup of the DRG, together suggested to the inventors that GM-284 likely acted on the Schwann cells, and that axonogenesis was a result of GM-284-mediated Schwann cell/neuron interactions. With this in mind, the inventors conducted a series of assays that demonstrated GM-284 induction of one or more Schwann-cell-derived soluble factors, the activity of which is recoverable in Schwann cell-GM-284 conditioned media (FIG. 4).

Furthermore, the inventors wanted to be sure that, if GM-284-mediated in vitro regeneration were Schwann-cell-dependent, there would be an immunophilin in the Schwann cell capable of binding GM-284. To this end, the inventors have shown the Schwann cell expression of the immunophilin, FKBP52, by both immunofluorescence and Western blot; using QTFS, the inventors have also shown that GM-284 binds to FKBP52 with an affinity similar to the parent compound FK506. Additionally, the inventors have demonstrated that GM-284 and FK506 are likely to bind to FKBP52 by similar mechanisms, as each ligand is able to compete for binding to the receptor. These data, taken together with the failure of GM-284 to upregulate the MEK1 cascade in dorsal root ganglia, strongly suggest that the inventors have identified a novel, Schwann-cell-mediated pathway that leads to neurite promotion in in vitro models of regeneration.

Dramatic changes in cell morphology, including axon outgrowth or secretion of factors in response to external stimuli, are almost invariably associated with alterations in gene expression. Therefore, the effects of GM-284 raised the likelihood that GM-284 was acting to alter Schwann cell gene expression directly. To test this possibility, the inventors took advantage of cDNA array technology—a recently-developed method that allows for simultaneous analysis of global changes in gene expression in cells or tissue under control and experimental conditions (Massimi et al., “Printing and Preparation of Slides for Microarray Analysis”, in Molecular Cloning—A Laboratory Manual, vol. 4, “Microarrays” chapter, Sambrook and Bowtell, eds. (Cold Spring Harbor, N.Y.: Cold Spring Harbor Press, 2002).

The inventors' comparisons of gene expression between control Schwann cells and Schwann cells treated with GM-284 for either 4 or 48 h confirm that GM-284 mediates gene expression in a temporal and sequential manner. The genes upregulated after 4 h of GM-284 treatment were distinct from those upregulated at 48 h. Notably, the number of genes induced by GM-284 increased with time (26 genes to 109 genes), suggesting that GM-284 initiates a cascade of gene expression. It is likely that further analysis of these cascades will extend insights into the molecular events surrounding nerve regeneration. Thus, cDNA-array analysis can be used to test the hypothesis that Schwann cell treatment with GM-284 initiates transcriptional changes, culminating in the secretion of one or more factors into the CM, which then induce axon outgrowth from purified sensory neurons.

The inventors' data support the idea that, via transcriptional alterations in a neighboring Schwann cell, GM-284 promotes an in vitro model of axonal regeneration. In order to extend these data to the in vivo system, the inventors turned to a nerve-crush model that they successfully utilized in the past to test the effects of Schwann-cell-expressed transcription factors in nerve regeneration. Following nerve crush, animals were randomized into either vehicle- or GM-284-treatment groups. The inventors documented the complete nature of the mechanical transection eletrophysiologically, and then followed the clinical and histological recovery over one month, with the animals receiving daily doses of GM-284 at 10 mg/kg.

Consistent with their in vitro data, the inventors observed enhanced axonal regeneration in the GM-284-treated animals. These data were remarkable in that the drug had no demonstrable effects on DRG neurons, even though there was a 3- to 4-fold increase in the size of myelinated axons following a month of GM-284 treatment. The complexity of analyzing the mechanisms of action of in vivo treatment prevents an absolute understanding of how GM-284, or any drug, functions in the whole animal. However, when taken together with the inventors' in vitro data, and with previous observations, discussed below, it appears that GM-284 is acting on the Schwann cell in vivo in a manner similar to its actions in vitro.

Notably, two of the genes upregulated by GM-284, SCIP and Brn-5 (both members of the POU family of transcription factors) have been shown by the inventors to be expressed in series as Schwann cells make the transition from promyelination to myelinating Schwann cells (SCIP) (Weinstein et al., Premature Schwann cell differentiation and hypermyelination in mice expressing a targeted antagonist of the POU transcription factor SCIP. Mol. Cell. Neurosci., 6:212-29, 1995), and the maintenance of the myelinating phenotype (Wu et al., The POU gene Brn-5 is induced by neuregulin and is restricted to myelinating Schwann cells. Mol. Cell Neurosci., 17:683-95, 2001) (Brn-5). In development, SCIP expression is transient, while Brn-5 expression is continuous in myelinating Schwann cells. Moreover, in the regenerated nerve, the expression of these genes is inverted in the distal nerve stump, such that myelinating Schwann cells continuously express SCIP, rather than Brn-5 (Scherer et al., Axons regulate Schwann cell expression of the POU transcription factor SCIP. J. Neurosci., 14(4):1930-42, 1994; Wu et al., The POU gene brn-5 is induced by neuregulin and is restricted to myelinating Schwann cells. Mol. Cell Neurosci., 17:683-95, 2001). Thus, at the molecular and transcriptional levels, the regenerated Schwann cell differs from the developing Schwann cell.

The inventors previously have shown that transgenic mice which harbor a mutation of the SCIP gene (termed ASCIP) such that the encoded protein retains the DNA-binding domain, but has a deletion of the NH₂-terminal regulatory domain, regenerate their peripheral nerves at an exceptionally accelerated rate (Gondre et al., Accelerated nerve regeneration mediated by Schwann cells expressing a mutant form of the POU protein SCIP. J. Cell Biol., 141:493-501, 1998). Moreover, while the ΔSCIP transgene expression is restricted to the Schwann cell, the activity it exerts in regeneration is both in cis (on itself) and in trans (on the axon). Crushing injury of the sciatic nerve of the ΔSCIP mice results in myelin hypertrophy. At the same time, the ΔSCIP Schwann cells act in trans, inducing axonal hypertrophy.

Despite the foregoing, it is almost a certainty that the designation of cis and trans, as used herein, is arbitrary: as axons grow, they are likely to act on the Schwann cells, providing a range of signals and support, and, similarly, the hyper-stimulated Schwann cells feed back on the axon in much the same way. This overstimulation of a given cascade in one half of the Schwann cell/axon unit is likely to result in overstimulation of the other half of the unit.

The appearance of the regenerated ASCIP sciatic nerve is virtually indistinguishable from the appearance of the regenerated, GM-284-treated sciatic nerve in wild-type animals (FIG. 7). In addition, GM-284 mediates DRG neuritogenesis in a neurotrophin-independent manner. The inventors have previously demonstrated that ΔSCIP Schwann cells also mediate DRG neuritogenesis in a neurotrophin-independent manner (Gondre et al., Accelerated nerve regeneration mediated by Schwann cells expressing a mutant form of the POU protein SCIP. J. Cell Biol., 141:493-501, 1998). In both cases, the response of the neuronal compartment in the Schwann cell/axon unit is dependent on signaling from the Schwann cell compartment of the unit.

Taken in toto, the data presented herein support the concept that GM-284 mediates activation of endogenous Schwann cell transcriptional cascades, altering one of the two components of the Schwann cell/axon unit. Additionally, alterations of these cascades in the Schwann cell represent the mechanism by which GM-284, and the other immunophilin ligands, act to promote peripheral nerve regeneration.

While the foregoing invention has been described in some detail for purposes of clarity and understanding, it will be appreciated by one skilled in the art, from a reading of the disclosure, that various changes in form and detail can be made without departing from the true scope of the invention in the appended claims.

What is claimed is:

1. A method for enhancing regeneration of a neurite in damaged nervous tissue, comprising contacting at least one Schwann cell adjacent to the neurite in the damaged nervous tissue with an amount of an immunophilin ligand effective to enhance regeneration of the neurite.

2. The method of claim 1, wherein the damaged nervous tissue comprises damaged peripheral neurons.

3. The method of claim 1, wherein the neurite is selected from the group consisting of a DRG neurite, an interneuron neurite, a motor neuron neurite, a peripheral neuron neurite, a sensory neuron neurite, and a neurite of the spinal cord.

4. The method of claim 1, wherein the immunophilin ligand is FK506 or an FK506 derivative.

5. The method of claim 4, wherein the FK506 derivative is nonimmunosuppressive.

6. The method of claim 5, wherein the nonimmunosuppressive FK506 derivative is GM-284.

7. The method of claim 1, wherein the contacting is effected in vitro.

8. The method of claim 1, wherein the contacting is effected in vivo in a subject.

9. The method of claim 8, wherein the contacting is effected in vivo in a subject by administering the immunophilin ligand to the subject.

10. The method of claim 9, wherein the immunophilin ligand is administered to the subject by oral administration, parenteral administration, sublingual administration, transdermal administration, or osmotic pump.

11. The method of claim 8, wherein the subject is a human.

12. The method of claim 11, wherein the human has nervous tissue degeneration.

13. The method of claim 12, wherein the nervous tissue degeneration is a peripheral neuropathy.

14. The method of claim 13, wherein the peripheral neuropathy is associated with a condition selected from the group consisting of acquired immune deficiency syndrome (AIDS)), acute or chronic inflammatory polyneuropathy, amyloidosis, amyotrophic lateral sclerosis (ALS), carpal tunnel syndrome, Charcot-Marie-Tooth disease, diabetes mellitus, diphtheria, Guillain-Barré syndrome, hereditary motor and sensory neuropathy (types I, II, or III), a hereditary neuropathy with liability to pressure palsy (HNPP), hypothyroidism, Lyme disease, leprosy, leukodystrophy, neurofibromatosis, nutritional deficiencies, peroneal muscular atrophy, peroneal nerve palsy, polio, polyarteritis nodosa, porphyria, postpolio syndrome, progressive bulbar palsy, Proteus syndrome, rheumatoid arthritis, radial nerve palsy, sarcoidosis, Sjögren's syndrome, systemic lupus erythematosus, spinal muscular atrophy, a toxic agent, trauma, ulnar nerve palsy, and uremia.

15. The method of claim 14, wherein the peripheral neuropathy is ALS or a hereditary peripheral neuropathy.

16. Use of an immunophilin ligand to regenerate a neurite in damaged nervous tissue, wherein at least one Schwann cell adjacent to the neurite in the damaged nervous tissue is contacted with an amount of the immunophilin ligand effective to enhance regeneration of the neurite.

17. A method for enhancing remyelination of a neurite in damaged nervous tissue, comprising contacting at least one Schwann cell adjacent to the neurite in the damaged nervous tissue with an amount of an immunophilin ligand effective to enhance remyelination of the neurite.

18. The method of claim 17, wherein the damaged nervous tissue comprises damaged peripheral neurons.

19. The method of claim 17, wherein the neurite is selected from the group consisting of a DRG neurite, an interneuron neurite, a motor neuron neurite, a peripheral neuron neurite, a sensory neuron neurite, and a neurite of the spinal cord.

20. The method of claim 17, wherein the immunophilin ligand is FK506 or an FK506 derivative.

21. The method of claim 20, wherein the FK506 derivative is nonimmunosuppressive.

22. The method of claim 21, wherein the nonimmunosuppressive FK506 derivative is GM-284.

23. The method of claim 17, wherein the contacting is effected in vitro.

24. The method of claim 17, wherein the contacting is effected in vivo in a subject.

25. The method of claim 24, wherein the contacting is effected in vivo in a subject by administering the immunophilin ligand to the subject.

26. The method of claim 25, wherein the immunophilin ligand is administered to the subject by oral administration, parenteral administration, sublingual administration, transdermal administration, or osmotic pump.

27. The method of claim 24, wherein the subject is a human.

28. The method of claim 27, wherein the human has nervous tissue degeneration.

29. The method of claim 28, wherein the nervous tissue degeneration is a peripheral neuropathy.

30. The method of claim 29, wherein the peripheral neuropathy is associated with a condition selected from the group consisting of acquired immune deficiency syndrome (AIDS)), acute or chronic inflammatory polyneuropathy, amyloidosis, amyotrophic lateral sclerosis (ALS), carpal tunnel syndrome, Charcot-Marie-Tooth disease, diabetes mellitus, diphtheria, Guillain-Barré syndrome, hereditary motor and sensory neuropathy (types I, II, or III), a hereditary neuropathy with liability to pressure palsy (HNPP), hypothyroidism, Lyme disease, leprosy, leukodystrophy, neurofibromatosis, nutritional deficiencies, peroneal muscular atrophy, peroneal nerve palsy, polio, polyarteritis nodosa, porphyria, postpolio syndrome, progressive bulbar palsy, Proteus syndrome, rheumatoid arthritis, radial nerve palsy, sarcoidosis, Sjögren's syndrome, systemic lupus erythematosus, spinal muscular atrophy, a toxic agent, trauma, ulnar nerve palsy, and uremia.

31. The method of claim 30, wherein the peripheral neuropathy is ALS or a hereditary peripheral neuropathy.

32. Use of an immunophilin ligand to enhance remyelination of a neurite in damaged nervous tissue, wherein at least one Schwann cell adjacent to the neurite in the damaged nervous tissue is contacted with an amount of the immunophilin ligand effective to enhance remyelination of the neurite.

33. A method for inducing hypermyelination of a neurite in nervous tissue, comprising contacting at least one Schwann cell adjacent to the neurite in the nervous tissue with an amount of an immunophilin ligand effective to induce hypermyelination of the neurite.

34. The method of claim 33, wherein the nervous tissue comprises damaged peripheral neurons.

35. The method of claim 33, wherein the neurite is selected from the group consisting of a DRG neurite, an interneuron neurite, a motor neuron neurite, a peripheral neuron neurite, a sensory neuron neurite, and a neurite of the spinal cord.

36. The method of claim 33, wherein the immunophilin ligand is FK506 or an FK506 derivative.

37. The method of claim 36, wherein the FK506 derivative is non immuno suppressive.

38. The method of claim 37, wherein the nonimmunosuppressive FK506 derivative is GM-284.

39. The method of claim 33, wherein the contacting is effected in vitro.

40. The method of claim 33, wherein the contacting is effected in vivo in a subject.

41. The method of claim 40, wherein the contacting is effected in vivo in a subject by administering the immunophilin ligand to the subject.

42. The method of claim 41, wherein the immunophilin ligand is administered to the subject by oral administration, parenteral administration, sublingual administration, transdermal administration, or osmotic pump.

43. The method of claim 40, wherein the subject is a human.

44. The method of claim 43, wherein the human has nervous tissue degeneration.

45. The method of claim 44, wherein the nervous tissue degeneration is a peripheral neuropathy.

46. The method of claim 45, wherein the peripheral neuropathy is associated with a condition selected from the group consisting of acquired immune deficiency syndrome (AIDS)), acute or chronic inflammatory polyneuropathy, amyloidosis, amyotrophic lateral sclerosis (ALS), carpal tunnel syndrome, Charcot-Marie-Tooth disease, diabetes mellitus, diphtheria, Guillain-Barré syndrome, hereditary motor and sensory neuropathy (types I, II, or III), a hereditary neuropathy with liability to pressure palsy (HNPP), hypothyroidism, Lyme disease, leprosy, leukodystrophy, neurofibromatosis, nutritional deficiencies, peroneal muscular atrophy, peroneal nerve palsy, polio, polyarteritis nodosa, porphyria, postpolio syndrome, progressive bulbar palsy, Proteus syndrome, rheumatoid arthritis, radial nerve palsy, sarcoidosis, Sjögren's syndrome, systemic lupus erythematosus, spinal muscular atrophy, a toxic agent, trauma, ulnar nerve palsy, and uremia.

47. The method of claim 46, wherein the peripheral neuropathy is ALS or a hereditary peripheral neuropathy.

48. Use of an immunophilin ligand to induce hypermyelination of a neurite in nervous tissue, wherein at least one Schwann cell adjacent to the neurite in the nervous tissue is contacted with an amount of the immunophilin ligand effective to induce hypermyelination of the neurite.

49. A pharmaceutical composition, comprising GM-284 and a pharmaceutically acceptable carrier.

50. A method for modulating gene expression in a Schwann cell, comprising contacting the Schwann cell with an amount of an immunophilin ligand effective to modulate gene expression in the Schwann cell.

51. The method of claim 50, wherein the immunophilin ligand is FK506 or an FK506 derivative.

52. The method of claim 51, wherein the FK506 derivative is nonimmunosuppressive.

53. The method of claim 52, wherein the nonimmunosuppressive FK506 derivative is GM-284.

54. The method of claim 50, wherein the contacting is effected in vitro.

55. The method of claim 50, wherein the contacting is effected in vivo in a subject.

56. The method of claim 55, wherein the contacting is effected in vivo in a subject by administering the immunophilin ligand to the subject.

57. The method of claim 56, wherein the immunophilin administered to the subject by oral administration, parenteral administration, sublingual administration, transdermal administration, or osmotic pump.

58. The method of claim 56, wherein the subject is a human.

59. The method of claim 58, wherein the human has a peripheral neuropathy.

60. The method of claim 59, wherein the peripheral neuropathy is associated with a condition selected from the group consisting of acquired immune deficiency syndrome (AIDS)), acute or chronic inflammatory polyneuropathy, amyloidosis, amyotrophic lateral sclerosis (ALS), carpal tunnel syndrome, Charcot-Marie-Tooth disease, diabetes mellitus, diphtheria, Guillain-Barré syndrome, hereditary motor and sensory neuropathy (types I, II, or III), a hereditary neuropathy with liability to pressure palsy (HNPP), hypothyroidism, Lyme disease, leprosy, leukodystrophy, neurofibromatosis, nutritional deficiencies, peroneal muscular atrophy, peroneal nerve palsy, polio, polyarteritis nodosa, porphyria, postpolio syndrome, progressive bulbar palsy, Proteus syndrome, rheumatoid arthritis, radial nerve palsy, sarcoidosis, Sjögren's syndrome, systemic lupus erythematosus, spinal muscular atrophy, a toxic agent, trauma, ulnar nerve palsy, and uremia.

61. The method of claim 60, wherein the peripheral neuropathy is ALS or a hereditary peripheral neuropathy.

62. Use of an immunophilin ligand to modulate gene expression in a Schwann cell, wherein the Schwann cell is contacted with an amount of the immunophilin ligand effective to modulate gene expression in the Schwann cell.

63. A method for treating a peripheral neuropathy in a subject in need of treatment, comprising modulating expression of a Schwann cell transcription factor in the subject, wherein the Schwann cell transcription factor is selected from the group consisting of SCIP and Brn-5.

64. The method of claim 63, wherein the peripheral neuropathy in the subject is treated by enhancing regeneration of at least one neurite in the subject.

65. The method of claim 63, wherein the peripheral neuropathy in the subject is treated by enhancing remyelination of at least one neurite in the subject.

66. The method of claim 63, wherein the peripheral neuropathy in the subject is treated by inducing hypermyelination of a neurite in nervous tissue in the subject.

67. The method of claim 63, wherein expression of a Schwann cell transcription factor is modulated in the subject by administering an immunophilin ligand to the subject.

68. The method of claim 67, wherein the immunophilin ligand is administered to the subject by oral administration, parenteral administration, sublingual administration, transdermal administration, or osmotic pump.

69. The method of claim 67, wherein the immunophilin ligand is administered to the subject in an amount effective to treat the peripheral neuropathy in the subject.

70. The method of claim 67, wherein the immunophilin ligand is FK506 or an FK506 derivative.

71. The method of claim 70, wherein the FK506 derivative is nonimmunosuppressive.

72. The method of claim 71, wherein the nonimmunosuppressive FK506 derivative is GM-284.

73. The method of claim 63, wherein the peripheral neuropathy is associated with a condition selected from the group consisting of acquired immune deficiency syndrome (AIDS)), acute or chronic inflammatory polyneuropathy, amyloidosis, amyotrophic lateral sclerosis (ALS), carpal tunnel syndrome, Charcot-Marie-Tooth disease, diabetes mellitus, diphtheria, Guillain-Barré syndrome, hereditary motor and sensory neuropathy (types I, II, or HI), a hereditary neuropathy with liability to pressure palsy (HNPP), hypothyroidism, Lyme disease, leprosy, leukodystrophy, neurofibromatosis, nutritional deficiencies, peroneal muscular atrophy, peroneal nerve palsy, polio, polyarteritis nodosa, porphyria, postpolio syndrome, progressive bulbar palsy, Proteus syndrome, rheumatoid arthritis, radial nerve palsy, sarcoidosis, Sjögren's syndrome, systemic lupus erythematosus, spinal muscular atrophy, a toxic agent, trauma, ulnar nerve palsy, and uremia.

74. The method of claim 73, wherein the peripheral neuropathy is ALS or a hereditary peripheral neuropathy.

75. A method for treating a peripheral neuropathy in a subject in need of treatment, comprising administering to the subject an amount of GM-284 effective to treat the peripheral neuropathy in the subject.

76. The method of claim 75, wherein the peripheral neuropathy is associated with a condition selected from the group consisting of acquired immune deficiency syndrome (AIDS)), acute or chronic inflammatory polyneuropathy, amyloidosis, amyotrophic lateral sclerosis (ALS), carpal tunnel syndrome, Charcot-Marie-Tooth disease, diabetes mellitus, diphtheria, Guillain-Barré syndrome, hereditary motor and sensory neuropathy (types I, II, or III), a hereditary neuropathy with liability to pressure palsy (HNPP), hypothyroidism, Lyme disease, leprosy, leukodystrophy, neurofibromatosis, nutritional deficiencies, peroneal muscular atrophy, peroneal nerve palsy, polio, polyarteritis nodosa, porphyria, postpolio syndrome, progressive bulbar palsy, Proteus syndrome, rheumatoid arthritis, radial nerve palsy, sarcoidosis, Sjögren's syndrome, systemic lupus erythematosus, spinal muscular atrophy, a toxic agent, trauma, ulnar nerve palsy, and uremia.

77. The method of claim 76, wherein the peripheral neuropathy is ALS or a hereditary peripheral neuropathy.

78. The method of claim 75, wherein GM-284 is administered to the subject by oral administration, parenteral administration, sublingual administration, transdermal administration, or osmotic pump.

79. Use of GM-284 to treat a peripheral neuropathy in a subject in need of treatment, wherein GM-284 is administered to the subject in an amount effective to treat the peripheral neuropathy in the subject.
